(19) United States
(12) Patent Application Publication
(10) Pub. No.: US 2025/0391518 A1
(43) Pub. Date: Dec. 25, 2025

(54) TRAINING AND UTILIZING COMPOUND GRAPH NEURAL NETWORKS TO GENERATE BIOLOGICAL ACTIVITY PREDICTIONS FROM INPUT CHEMICAL COMPOUNDS

(71) Applicant: Recursion Pharmaceuticals, Inc., Salt Lake City, UT (US)

(72) Inventors: Dominique BEAINI, Kirkland (CA); Farimah RAMEZAN POURSAFAEI, Montreal (CA); Jan Frederik WENKEL, Montreal (CA); Maciej SYPETKOWSKI, Warsaw (PL)

(21) Appl. No.: 18/750,813

(22) Filed: Jun. 21, 2024

(51) Int. Cl. G16C 20/70; G16C 20/30

(52) U.S. Cl. CPC G16C 20/70; G16C 20/30

(57) ABSTRACT

The present disclosure relates to systems, non-transitory computer-readable media, and methods for training and utilizing compound graph neural networks to generate graph representations of input compounds, extract fingerprints, and utilize the fingerprints to generate biological activity predictions relating to the input compounds. For example, the disclosed systems can train a compound graph neural network to generate a graph representation of an input compound. Additionally, the disclosed systems can extract a fingerprint of the graph representation and utilize the fingerprint to make a biological activity prediction for the input compound. In some cases, the disclosed systems can compare the biological activity prediction with a ground truth for the input compound and utilize the comparison to finetune the parameters of the compound graph neural network. Furthermore, in some cases, the disclosed systems can ensemble fingerprints generated from multiple graph representations to generate the biological activity prediction.

### BACKGROUND

Recent years have seen significant developments in hardware and software platforms for training and utilizing machine learning models in conjunction with computer-implemented pharmaceutical discovery systems. For example, conventional systems utilize large volumes of training to analyze chemical compounds and generate various predictions. Despite these recent advances, conventional systems suffer from a number of technical deficiencies, particularly with regard to accuracy, efficiency, and operational inflexibility in implementing machine learning technologies. These deficiencies are particularly profound when it comes to the computational resources required to train new models.

### SUMMARY

Embodiments of the present disclosure provide benefits and/or solve one or more of the foregoing or other problems in the art with systems, non-transitory computer-readable media, and methods for utilizing machine learning models to extract fingerprints from graph representations of an input compound and utilizing the fingerprints to make biological activity predictions for the input compound. For example, the disclosed systems generate a graph representation of an input chemical compound, wherein individual molecules of the input compound are represented as nodes of the graph representation, and chemical bonds between individual molecules are represented as edges of the graph representation. The disclosed systems can utilize a compound graph neural network to analyze the graph representation via one or more pre-trained prediction heads to generate a variety of predictions for novel tasks, such as chemical activity predictions, compound program predictions, phenomic embedding predictions, and/or transcriptomic predictions.

In addition, in one or more implementations, the disclosed systems also train and utilize machine learning models through unique finetuning approaches that extract fingerprints from pre-trained prediction heads and/or existing trained machine learning models and repurpose these feature representations for generating additional predictions for an input compound. For example, the disclosed systems can utilize fingerprints extracted from one or more layers of an existing pre-trained prediction head that has been trained for an alternative task. Similarly, the disclosed systems can utilize ensemble fingerprinting by extracting fingerprints from separately trained machine learning models and combining these fingerprints for an alternative task. By utilize these fingerprinting and/or ensemble fingerprinting models, the disclosed systems can efficiently finetune existing models to flexibly transition to generating new biological activity predictions. Moreover, by utilizing these finetuned machine learning models to analyze input compounds, the disclosed can generate accurate biological activity predictions based on the learned interactions represented in feature representations of pre-trained task heads trained on previous tasks.

Additional features and advantages of one or more embodiments of the present disclosure are outlined in the description which follows, and in part will be obvious from the description, or may be learned by the practice of such example embodiments.

### BRIEF DESCRIPTION OF THE DRAWINGS

The detailed description provides one or more embodiments with additional specificity and detail through the use of the accompanying drawings, as briefly described below.

FIG. 1 illustrates a molecular graph prediction system extracting fingerprints of an input compound for generating biological activity predictions in accordance with one or more embodiments.

FIG. 2 illustrates an example architecture of a compound graph neural network in accordance with one or more embodiments.

FIG. 3 illustrates a molecular graph prediction system extracting fingerprints in accordance with one or more embodiments.

FIG. 4 illustrates the molecular graph prediction system extracting fingerprints from multiple sub-graph neural networks in accordance with one or more embodiments.

FIG. 5 illustrates the molecular graph prediction system extracting a fingerprint from one or more layers of a pre-trained prediction head in accordance with one or more embodiments.

FIG. 6 illustrates an implementation of the molecular graph prediction system generating various biological activity predictions in accordance with one or more embodiments.

FIG. 7 illustrates a graphical representation of experimental results achieved by an experimental implementation of the molecular graph prediction system in accordance with one or more embodiments.

FIG. 8 illustrates a method for extracting fingerprints in accordance with one or more embodiments.

FIG. 9 illustrates an example environment of the molecular graph prediction system in accordance with one or more embodiments.

FIG. 10 illustrates a block diagram of a computing device for implementing one or more embodiments.

### DETAILED DESCRIPTION

This disclosure describes one or more embodiments of a molecular graph prediction system that trains and utilizes a compound graph neural network architecture to generate biological activity predictions from input compounds. For example, the molecular graph prediction system can utilize a compound graph neural network to analyze an input compound and generate a variety of predictions for novel tasks, such as chemical activity predictions, compound program predictions, phenomic embedding predictions, and/or transcriptomic predictions. Moreover, the molecular graph prediction system can also extract fingerprints from pre-trained prediction heads to finetune and implement a compound graph neural network for generating additional or alternative predictions. For example, the molecular graph prediction system can initially train a compound graph neural network to generate a variety of quantum physics, chemistry, or biology tasks utilizing a first set of pre-trained prediction heads. The molecular graph prediction system can then finetune the compound graph neural network by extracting fingerprints from these pre-trained prediction heads (and/or extracting fingerprints from other pre-trained models) and utilize additional, efficient neural network layers to generate predictions for additional tasks. In this manner, the disclosed systems can train and utilize compound graph neural networks to flexibly transform and utilize input compounds to generate accurate biological activity predictions.

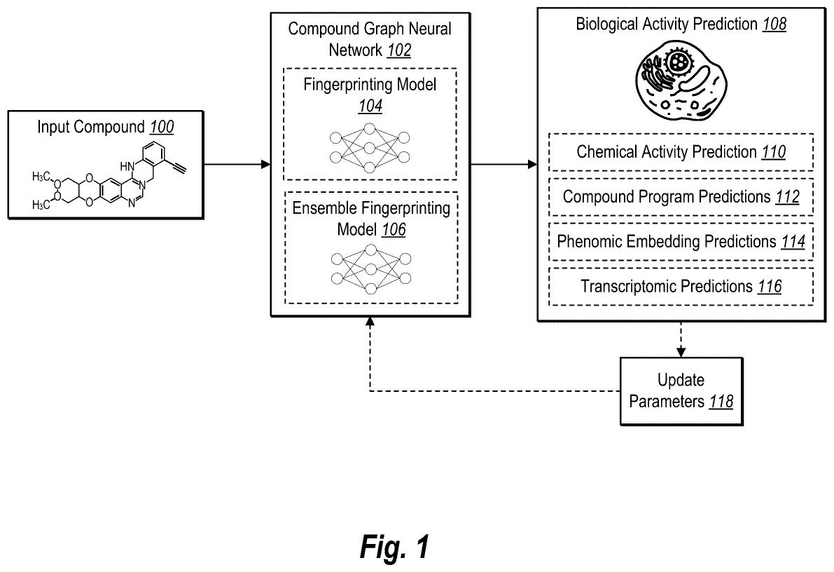

As just mentioned, the molecular graph prediction system can train and utilize a compound graph neural network to generate biological activity predictions. For example, FIG. 1 illustrates the molecular graph prediction system generating biological activity predictions 108 from an input compound 100 utilizing a compound graph neural network 102 in accordance with one or more embodiments.

Specifically, as illustrated in FIG. 1, the molecular graph prediction system receives, identifies, and/or generates an input compound 100. For example, the molecular graph prediction system can generate a digital representation of a chemical compound. To illustrate, the molecular graph prediction system can receive a query from a client device identifying the input compound 100. The molecular graph prediction system can then identify features of the input compound 100 and transform the input compound 100 into a digital representation. For instance, the molecular graph prediction system can generate a representation of the atoms, bonds, structure, properties, or other features of the input compound 100.

In one or more implementations, the molecular graph prediction system utilizes a compound graph neural network 102 to generate a graph representation of the input compound 100. Specifically, the molecular graph prediction system constructs a graph representation that includes node features and edge features. Specifically, the molecular graph prediction system structures the graph representation such that the node features correspond to molecules of the input compound and the edge features correspond to bonds between the molecules of the input compound.

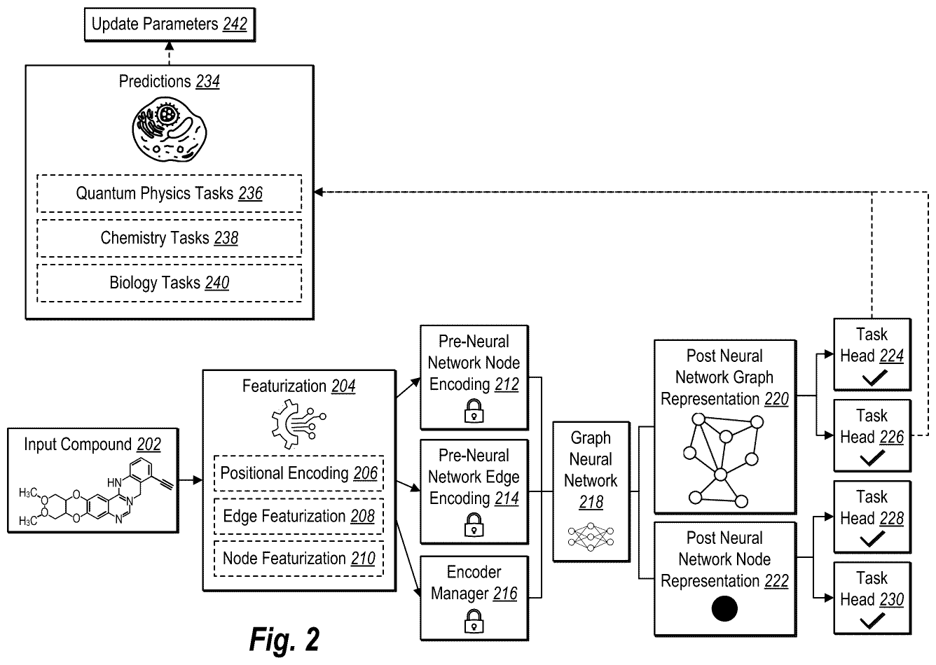

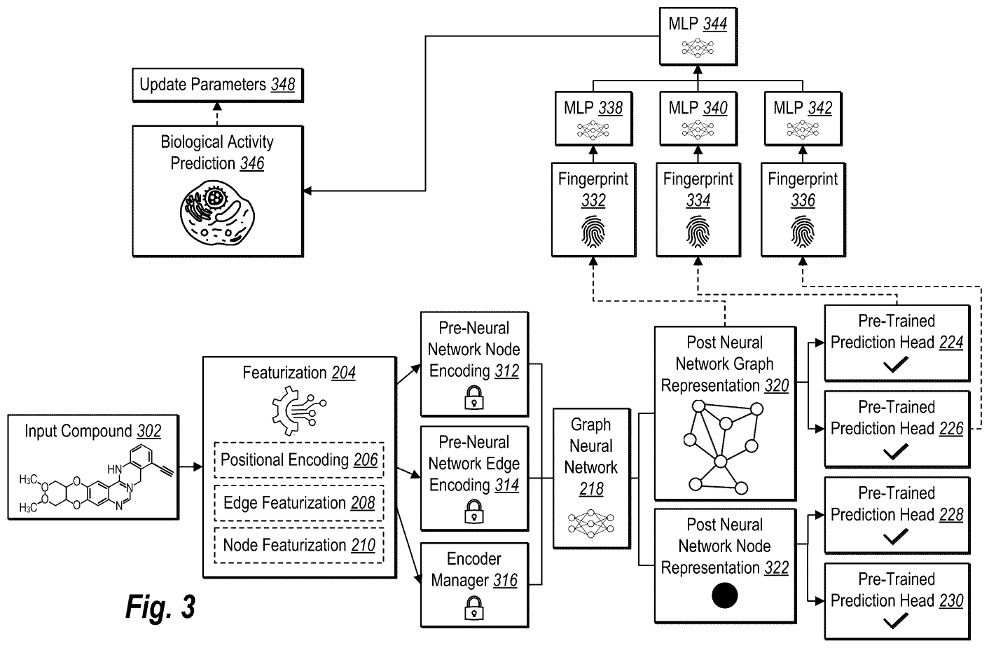

In one or more implementations, the compound graph neural network 102 includes multiple prediction heads (e.g., pretrained-prediction heads). For example, the molecular graph prediction system performs an initial training of the compound graph neural network 102 by utilizing multiple prediction heads to generate predictions for multiple training tasks. In this manner, the molecular graph prediction system trains the compound graph neural network 102 on a diversity of tasks to learn a complex feature space that represents variety of physical and biological interactions. In one or more implementations, the molecular graph prediction system trains multiple compound graph neural networks (e.g., with different prediction heads and/or different training data). Additional detail regarding this initial training of one or more compound graph neural network architecture for multiple prediction tasks is provided below (e.g., in relation to FIGS. 2-3B).

After this initial training, as shown in FIG. 1, the molecular graph prediction system can utilize a variety of models (e.g., a fingerprinting model 104 and/or an ensemble fingerprinting model 106) to further finetune and/or implement the compound graph neural network 102. In particular, as shown, the molecular graph prediction system utilizes a fingerprinting model 104 to finetune and generate predictions for a new task utilizing the compound graph neural network 102.

For instance, the molecular graph prediction system can utilize the fingerprinting model 104 to extract a fingerprint from one or more of the pre-trained prediction heads of the compound graph neural network 102. For example, in one or more implementations, the compound graph neural network 102 utilizes the compound graph neural network 102 and a pre-trained prediction head to generate a vector representation (e.g., a fingerprint) from the graph representation of the input compound 100. The molecular graph prediction system utilizes this fingerprint from the pre-trained prediction head to finetune the compound graph neural network 102 for an alternate task and/or to generate a prediction for an alternate task.

Indeed, in one or more implementations, the molecular graph prediction system extracts a plurality of fingerprints (e.g., from multiple pre-trained prediction heads) and processes the plurality of fingerprints through additional neural networks (e.g., lightweight multi-layer perceptrons with fewer parameters) to generate a prediction for an additional task. For instance, the molecular graph prediction system processes a first fingerprint from a first pre-trained prediction head through a neural network to generate a first fingerprint representation and process a second fingerprint from a second pre-trained prediction head through another neural network to generate a second fingerprint representation. The molecular graph prediction system then combines the first fingerprint representation and the second fingerprint representation utilizing a further neural network to generate a prediction for an additional task. Additional detail regarding extracting and utilizing fingerprints for finetuning or implementing a compound graph neural network is provided below (e.g., in relation to FIGS. 3-6).

As shown in FIG. 1, the molecular graph prediction system can also utilize an ensemble fingerprinting model 106 finetune and/or implement the compound graph neural network 102. For example, rather than utilizing pre-trained prediction heads jointly trained from a common model, the molecular graph prediction system can utilize pre-trained prediction heads from separately trained networks to finetune and implement the compound graph neural network 102.

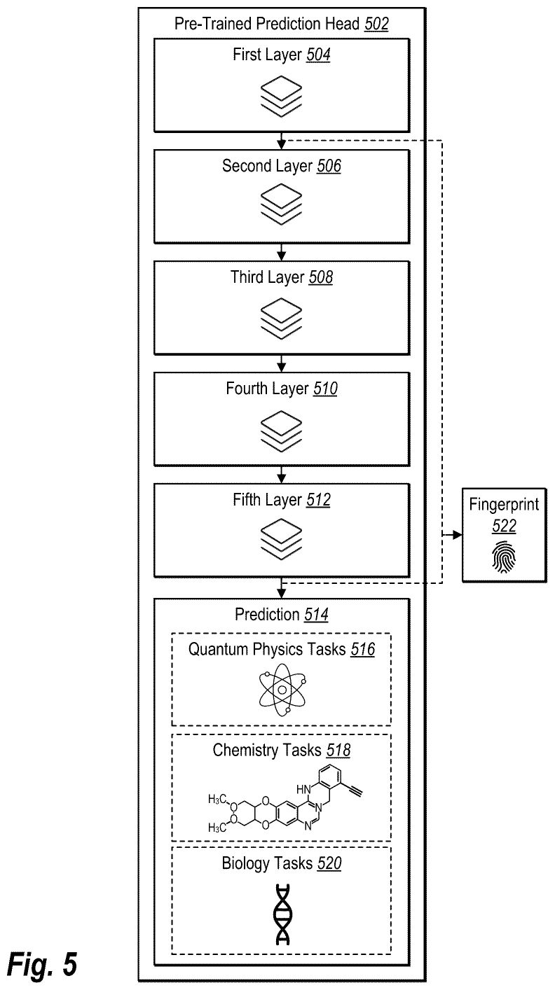

To illustrate, the molecular graph prediction system can utilize a first sub-graph neural network to generate a first graph representation of the input compound 100 and utilize a first prediction head of the first sub-graph neural network to generate a first vector representation (e.g., a first fingerprint). The molecular graph prediction system can utilize a second sub-graph neural network to generate a second graph representation of the input compound 100 and utilize a second prediction head of the second sub-graph neural network to generate a second vector representation (e.g., a second fingerprint). Thereafter, the molecular graph prediction system can combine the first fingerprint and the second fingerprint (utilizing additional neural networks) to generate a prediction for an additional task. Additional detail regarding the molecular graph prediction system utilizing an ensemble fingerprinting model is provided below (e.g., in relation to FIG. 5).

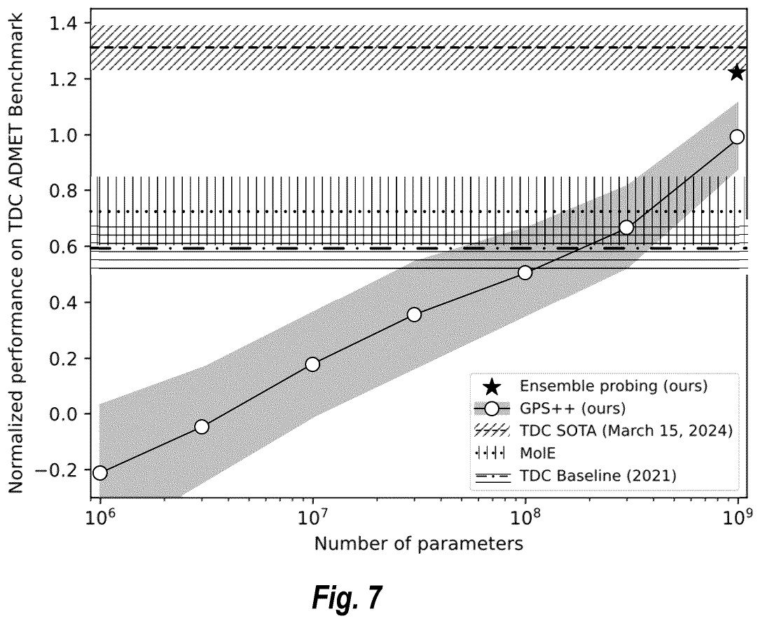

Indeed, as shown in FIG. 1, the molecular graph prediction system utilizes the fingerprinting model 104 or the ensemble fingerprinting model 106 of the compound graph neural network 102 to generate a biological activity prediction 108. Indeed, the molecular graph prediction system can utilize the compound graph neural network 102 to generate a variety of novel predictions, such as a chemical activity prediction 110 (e.g., a level of activity or interaction of a compound within a cell or body), a compound program prediction 112, a phenomic embedding prediction 114, or a transcriptomic prediction 116, among others. Additional information regarding the molecular graph prediction system generating biological activity predictions is provided below (e.g., in relation to FIG. 7).

As shown in FIG. 1, the molecular graph prediction system can also perform an (optional) act 118 of updating parameters of the compound graph neural network 102. For example, the molecular graph prediction system can compare a biological activity prediction 108 with a known biological activity of the input compound 100 (e.g., a ground truth), and update the parameters of the compound graph neural network 102 based on the comparison. For example, the molecular graph prediction system can compare the biological activity prediction 108 to a dataset containing known aspects of the biological activity of the input compound. The molecular graph prediction system can utilize various techniques to update the parameters of the compound graph neural network 102, such as backpropagation and gradient descent.

Although the act 118 relates to training/finetuning the compound graph neural network 102, the molecular graph prediction system can also utilize the molecular graph prediction system after training to generate biological activity predictions. Indeed, by utilizing fingerprints from a variety of fingerprints from pre-trained prediction heads together with finetuned neural networks for further processing those fingerprints, the molecular graph prediction system can more accurately generate bioactivity predictions.

Although not illustrated in FIG. 1, the molecular graph prediction system can utilize a compound graph neural network for a variety of additional purposes. For example, the molecular graph prediction system can utilize the compound graph neural network in conjunction with a generative model. Indeed, because the compound graph neural network can learn interconnected features for a variety of different prediction tasks, the molecular graph prediction system can utilize the compound graph neural network as part of a generative model for generating compounds (e.g., generating new/novel compounds, completing compounds, and/or modifying input compounds).

Similarly, in one or more implementations, the molecular graph prediction system utilizes feature representations from the compound graph neural network to determine similarities between compounds. For example, the molecular graph prediction system can compare fingerprints (e.g., feature vectors from one or more layers of the compound graph neural network) in a shared feature space and determine a measure of similarity (e.g., a distance measure within the feature space or a cosine similarity). The molecular graph prediction system can then utilize the measure of similarity to identify similar compounds. For example, the molecular graph prediction system can perform similarity screening for large compound libraries that contain millions or billions of molecules to identify those molecules that are similar to a particular query compound.

Furthermore, as new data is discovered (e.g., additional assays are performed) the molecular graph prediction system can automatically finetune the compound graph neural network to accommodate the new data. For example, the molecular graph prediction system can extract previous fingerprints generated for compounds and utilize those existing fingerprints to finetune new neural networks (e.g., new MLPs) to generate new predictions based on the new data. Thus, the molecular graph prediction system can iteratively finetune for new tasks based on previously learned features from other pre-trained prediction heads. Further, the molecular graph prediction system can utilize one or more additional machine learning models and/or updated data repositories to train and/or finetune parameters of the compound graph neural network. Moreover, as the molecular graph prediction system receives new data into a data repository or folder, the molecular graph prediction system can automatically finetune the model and save a checkpoint into the data repository.

As mentioned above, conventional systems suffer from a number of technical deficiencies with regard to implementing computing devices. For example, conventional systems often generate inaccurate machine learning predictions. Indeed, although conventional systems can utilize machine learning models to generate predictions, such predictions are often inaccurate because conventional systems utilize architectures and training approaches that undermine prediction accuracy. For example, conventional systems often generate predictions utilizing architectures trained for a single prediction task. Although this approach can generate predicted results, conventional systems are often plagued by imprecise and inaccurate machine learning outputs due to the underlying architecture and training processes.

Furthermore, conventional systems are often inefficient. For example, conventional systems often utilize significant computational resources in training individual machine learning models for generating particular predictions. This duplicative approach of learning parameters for models in generating different predictions utilizes excessive memory, processing power, and time of implementing computing devices. This is especially true in building large neural networks with millions of different learned parameters. Accordingly, conventional systems are often inefficient in training models and generating machine learning predictions.

Conventional systems are also operationally inflexible. For example, conventional systems generally develop models focused on individual predictive tasks. This leads to system rigidity in that conventional systems cannot easily pivot to new predictive tasks without expending significant time and computational resources. In addition, conventional models trained on any particular task are generally limited to learning from the underlying feature space corresponding to that task. This rigidity undermines the flexibility of models in being able to consider other biological interactions or feature spaces in generating predictions. It also impedes conventional systems from applying their models to new and novel predictive tasks.

As suggested by the foregoing discussion, the molecular graph prediction system provides a variety of technical advantages relative to conventional systems. For example, the molecular graph prediction system can utilize a compound graph neural network architecture trained on a plurality of different predictive tasks to model interactivity across a variety of biological activity features. For instance, the molecular graph prediction system can train a compound graph neural network on quantum physics tasks, chemistry tasks, and biology tasks simultaneously to learn information about how a molecule works across a variety of domains. Furthermore, the molecular graph prediction system can utilize a fingerprinting model or fingerprinting ensemble model to finetune models to generate accurate predictions for novel tasks based on vector representations from pre-trained prediction heads. Thus, the molecular graph prediction system can build and implement compound graph neural networks that generate accurate biological activity predictions.

In addition to accuracy improvements, in some embodiments, the molecular graph prediction system improves efficiency relative to conventional systems. Indeed, as mentioned, the molecular graph prediction system can efficiently finetune pre-trained models utilizing a fingerprinting model and/or ensemble fingerprinting model. Indeed, by extracting fingerprints from pre-trained prediction heads, the molecular graph prediction system can efficiently translate the learned model intelligence from a first predictive task to a novel predictive task. Not only does this approach incorporate the intelligence of the learned feature space for the previously trained biological activity prediction task, but this approach also significantly reduces time, memory, and computing resources needed to build a model for a new predictive task (e.g., in re-training neural networks with millions of different parameters or more). Moreover, as described in greater detail below, in some implementations, the molecular graph prediction system reuses previously generated fingerprints (e.g., stored in a fingerprint database) from a pre-trained prediction head to learn parameters for a new predictive task, further reducing computing resources needed to develop new predictive models.

Relatedly, in some embodiments, the molecular graph prediction system improves upon operational flexibility. Indeed, as just mentioned, the molecular graph prediction system can finetune pre-trained prediction heads utilizing a fingerprinting model and/or ensemble fingerprinting model to flexibly pivot existing predictive models to new predictive tasks. Indeed, the molecular graph prediction system can flexibly modify one or more existing graph neural networks trained on various biological activity predictive tasks and generate a new model that retains underlying intelligence of the pre-trained predictive heads. In addition, the molecular graph prediction system can flexibly generate new biological activity predictions utilizing a compound graph neural network. Indeed, as discussed in greater detail below, the molecular graph prediction system can apply the architecture of a compound graph neural network to generate new biological activity predictions from a query compound, including phenomic embedding predictions, transcriptomic predictions, compound program predictions, protein binding predictions, toxicity (or other ADMET property predictions), and/or other chemical activity predictions. Thus, the molecular graph prediction system allows implementing computing devices to utilize a compound graph neural network architecture to flexibly generate new and improved biological activity predictions.

As just mentioned, in one or more implementations, the molecular graph prediction system can initially train a compound graph neural network architecture to analyze input compounds and generate predictions. The molecular graph prediction system can then finetune a compound graph neural network for alternative tasks. For example, FIG. 2 illustrates initially training a compound graph neural network in accordance with one or more embodiments and FIG. 3 illustrates finetuning a compound graph neural network utilizing a fingerprinting model in accordance with one or more embodiments.

As used herein, the term “machine learning model” includes a computer algorithm or a collection of computer algorithms that can be trained and/or tuned based on inputs to approximate unknown functions. For example, a machine learning model can include a computer algorithm with branches, weights, or parameters that changed based on training data to improve for a particular task. Thus, a machine learning model can utilize one or more learning techniques (e.g., supervised or unsupervised learning) to improve in accuracy and/or effectiveness. Example machine learning models include various types of decision trees (e.g., gradient boost models), support vector machines, Bayesian networks, random forest models, or neural networks (e.g., deep neural networks, generative adversarial neural networks, convolutional neural networks, recurrent neural networks, or diffusion neural networks). Similarly, as used herein, a neural network refers to a machine learning model of interconnected nodes (or neurons) organized into layers. A neural network can include parameters or weights between neurons that are adjusted during training to minimize the error (or measure of loss) in generating predictions. Moreover, a graph neural network refers to a type of neural network designed to process data represented as graphs, where nodes represent entities and edges represent relationships between them.

As used herein, the term “compound graph neural network” refers to a neural network that utilizes a graph architecture to generate predictions regarding a compound. For example, a compound graph neural network includes a model that generates a graph representation of an input compound and utilizes the graph representation to make one or more biological activity predictions for the input compound based on one or more components of the graph representation.

For example, FIG. 2 illustrates a compound graph neural network that generates unique encodings of an input compound 202, processes the encodings utilizing a graph neural network 218 to generate a post neural network graph representation 220 and post neural network node representation(s) 222, and then utilizes one or more task heads (e.g., task head 224, task head 226, task head 228, or task head 230) to generate predictions 234 from the post neural network graph representation 220. The molecular graph prediction system then updates parameters to train the compound graph neural network (e.g., by comparing the predictions 234 with one or more ground truth observations).

As shown in FIG. 2, in some embodiments, the molecular graph prediction system can utilize a compound graph neural network to generate a post neural network graph representation 220 for an input compound 202 (e.g., the input compound 100 of FIG. 1). As mentioned previously, the molecular graph prediction system can receive the input compound 100 (e.g., from a user input of a query via a client device) and generate a digital representation of the input compound 100. The molecular graph prediction system can generate a variety of digital representations in a variety of formats, including Simplified Molecular Input Line Entry System (SMILES), SMILES Arbitrary Target Specification (SMARTS), International Chemical Identifier (InChI), InChIKey, Molecular 2D/3D File Format (MOL2), Protein Data Bank Format (PDB), RDKit, XYZ Files, Canonical SMILES, or Tensor Representations, among others. In some implementations, the digital representation of the input compound 100 includes vector representations of various compound features, such as three-dimensional features, atomic features, chemical properties, bonding features, or other features.

Indeed, as illustrated, the molecular graph prediction system can perform an act 204 of featurization on the input compound 202. In particular, the molecular graph prediction system can perform the act 204 and analyze these features utilizing one or more networks (e.g., multi-layer perceptrons) to generate various representations for analysis by the graph neural network 218. For instance, the molecular graph prediction system generates a pre-neural network node encoding 212 and a pre-neural network edge encoding 214. Furthermore, the molecular graph prediction system utilizes an encoder manager 216 to generate positional and structure feature representations for analysis by the graph neural network 218.

Specifically, the molecular graph prediction system can perform the act 206 of positional encoding for the input compound 202 to generate a representation of the spatial position and/structure of each atom and/or bond in the input compound 202. For instance, the molecular graph prediction system can analyze various features, such as Laplacian, eigenvector, Laplacian eigenvalues, and/or other positional encodings, (e.g., that reflect different positional vectors). The molecular graph prediction system can also perform analysis to determine connectivity. For example, the molecular graph prediction system can perform a random walk of the compound (e.g., and extract connectivity between atoms/nodes) that reflect the structure of the graph. Indeed, unlike text graphs (where a position for each word is generally known) in a compound graph the position for nodes is not readily identifiable.

In one or more implementations, the molecular graph prediction system can perform the act 208 of edge featurization to generate representations (e.g., one or more edge feature vectors) of the bonds between molecules of the input compound 202. Specifically, the molecular graph prediction system can perform act 208 edge featurization to represent information such as attributes of the bonds (e.g., bond type, aromaticity, stereochemistry, or numerical features such as bond length/angle) or contextual information (e.g., features of the bond derived from the properties of the connected atoms) in a feature vector.

As illustrated, the molecular graph prediction system can perform an act 210 of node featurization to generate representations (e.g., one or more feature vectors) of the atoms in the input compound 202. Specifically, the molecular graph prediction system can perform an act 210 of node featurization to represent information such as atom attributes (e.g., atomic number, partial charge, hybridization state, aromaticity, formal charge), local structural information (e.g., types and properties of neighboring atoms and bonds), and positional information (e.g., spatial coordinates representing the atom's location in three-dimensional space).

As illustrated, the molecular graph prediction system can utilize various features/encodings resulting from the act 204 generate a pre-neural network node encoding 212 and a pre-neural network edge encoding 214. For example, the molecular graph prediction system can utilize one or more pre-neural networks to encode the node features of the input compound 202 and represent them in the pre-neural network node encoding 212. Specifically, the molecular graph prediction system can utilize a first MLP encoder (e.g., a neural network encoder) to encode node features of the input compound 202 (e.g., atom number, mass, valence, etc.). Similarly, the molecular graph prediction system can utilize a second MLP encoder to encode the edge features of the input compound 202 (e.g., bond number, stereo, etc.). The molecular graph prediction system can utilize a third MLP encoder to encode the graph features of the input compound 202 (e.g., total mass, total charge, etc.). The molecular graph prediction system can utilize a gaussian kernel encoder to encode conformer features of the input compound 202 (e.g., 3D positions, energy, etc.).

As shown, the molecular graph prediction system can utilize an encoder manager 216 to determine the structure around the node (e.g., to generate a number or ordering for the nodes of the graph). The encoder manager 216 can include a variety of encoding models (e.g., multi-layer perceptrons or other neural networks) to generate structural feature representations corresponding to the nodes. For example, the molecular graph prediction system can utilize a Laplacian encoder and a SignNet encoder to encode Laplacian eigenvectors and eigenvalues representative of physical properties and structural elements of the input compound 202. The molecular graph prediction system can utilize a fourth MLP encoder to encode a representation with structural elements of the input compound. The molecular graph prediction system can utilize a fifth MLP encoder to encode the shortest path distance for the structural elements of the input compound 202.

Indeed, as shown, the molecular graph prediction system can utilize an encoder manager 216 to manage properties of the pre-neural network node encodings 212. For example, the molecular graph prediction system can utilize the encoder manager 216 to assign numbers to pre-neural network node encodings 212 (e.g., in a linear manner). The molecular graph prediction system utilizes the encoder manager 216 to increase the expressivity of the graph neural network 218 by providing additional information about the input compound 202.

In some embodiments, the molecular graph prediction system can combine the pre-neural network node encoding 212, the pre-neural network edge encoding 214, and feature representations generated by the encoder manager 216. Specifically, the molecular graph prediction system can combine the chemical features of the input compound (e.g., node features, edge features, graph features, and conformer features) and the physical properties and structural elements of the input compound (e.g., the Laplacian eigenvectors and eigenvalues, the representation with structural elements, and the shortest path distance). The molecular graph prediction system can utilize a variety of methods to perform this action. For example, the molecular graph prediction system can pool the pre-neural network node encodings 212 and pre-neural network edge encodings 214 by key. The molecular graph prediction system can group elements of the pre-neural network node encodings 212 and the pre-neural network edge encodings 214 into groups according to a shared key or identifier. Thereafter, the molecular graph prediction system can aggregate information within each group to produce a single output representation. As mentioned, the molecular graph prediction system can utilize keys in the input features and pool by keys corresponding to the feature vectors. Thus, the various MLPs described above can each generate an output feature vector or encoding. The molecular graph prediction system can pool these vectors/encodings by key. In other words, the molecular graph prediction system assigns matching input keys to both the features and the encoders, then pools the outputs according to the output keys. The molecular graph prediction system can utilize a variety of techniques to perform the aggregation, including averaging, pooling, max-pooling, or weighted pooling, among others.

In some embodiments, after combining the pre-neural network node encodings 212 and pre-neural network edge encodings 214, the molecular graph prediction system can generate a graph dictionary. In particular, the molecular graph prediction system can generate the graph dictionary to include four representations from the pre-neural network node encodings 212, pre-neural network edge encodings 214, and feature representations from the encoder manager 216. Specifically, the molecular graph prediction system can generate node features, edge features, graph features and attention bias. The molecular graph prediction system can utilize node features to represent the maximum number of nodes corresponding to atoms of the input compound 202 and a first hidden feature representation. The molecular graph prediction system can utilize edge features to represent a number of edges corresponding to bonds of the input compound 202 and a second hidden feature representation. The molecular graph prediction system can utilize graph features to represent graphs corresponding to the input compound 202 and a third hidden feature representation. The molecular graph prediction system can utilize attention bias to represent the number of graphs corresponding to the input compound 202, a first number of nodes corresponding to atoms of the input compound 202, a second number of nodes corresponding to atoms of the input compound 202, and a fourth hidden feature representation. For example, the molecular graph prediction system can utilize attention bias to represent node pairs features for nodes and edges (e.g., a source node and a destination node for each edge feature). Thus, the attention bis can reflect connectivity of atoms for later processing by the graph neural network 218 (e.g., a transformer of the graph neural network 218).

As shown in FIG. 2, after generating the pre-neural network node encodings 212 and pre-neural network edge encodings 214, and any additional information supplemented by the encoder manager 216, the molecular graph prediction system can utilize a graph neural network 218 to generate one or more post neural network node representations 222. The graph neural network 218 can include a variety of layers, including a transformer network, a message passing neural network, a graph convolutional network, a pattern agnostic neural network, or a graph isomorphism network, among others. Specifically, the molecular graph prediction system can utilize the graph neural network 218 to generate the post neural network node representation(s) 222 for the input compound 202. Post neural network node representation(s) 222 can corresponding to one or more atoms of the input compound 202.

Additionally, as shown in FIG. 2, the molecular graph prediction system can utilize the graph neural network 218 to generate a post neural network graph representation 220 (e.g., a graph representation of the input compound 202). Specifically, the molecular graph prediction system can utilize the pre-neural network node encodings 212, pre-neural network edge encodings, and additional information supplemented by the encoder manager 216 to generate the post neural network graph representation 220. In some embodiments, the molecular graph prediction system can generate the post neural network graph representation 220 by utilizing a pooling layer to combine the post neural network node representation(s) 222 with edge features to generate the post neural network graph representation 220.

As used herein, the term “graph representation” refers to an embedding or digital representation of an input compound generated via a graph neural network (e.g., reflecting edges and/or nodes of a graph). For example, a graph representation can include a feature vector or other representation that reflects nodes that correspond to atoms of the input compound and edges that correspond to bonds between atoms of the input compound. In one or more implementations, the molecular graph prediction system generates a graph representation utilizing a graph neural network from edge features and node features corresponding to an input compound. Thus, in some implementations, a graph representation includes the post neural network graph representation 220 (and/or the post neural network node representations 222).

In some embodiments, the molecular graph prediction system can utilize a light-weight neural network (e.g., an MLP) to process the post neural network graph representation 220 and/or the post neural network node representation(s) 222 into a format suitable for receipt and use by one or more task heads (e.g., task head 224, task head 226, task head 228, or task head 230). For example, the molecular graph prediction system can utilize the MLP (e.g., a graph output network) to transform the post neural network graph representation 220 or post neural network node representation(s) 222 into a high-dimensional feature representation. The molecular graph prediction system can provide the high-dimensional feature representation to a task head (e.g., task head 224, task head 226, task head 228) and cause the task head to utilize the high-dimensional feature representation to perform a task (e.g., generate a prediction).

As used herein, the term task head or “prediction head” refers to a collection of neural network layers utilized to generate a prediction (or perform a task). For example, a prediction head can include a sub-component of a graph neural network that analyzes input features (e.g., a graph representation of a compound) to generate a prediction. As mentioned, a compound graph neural network can have a variety of task heads or prediction heads that generate different types of predictions.

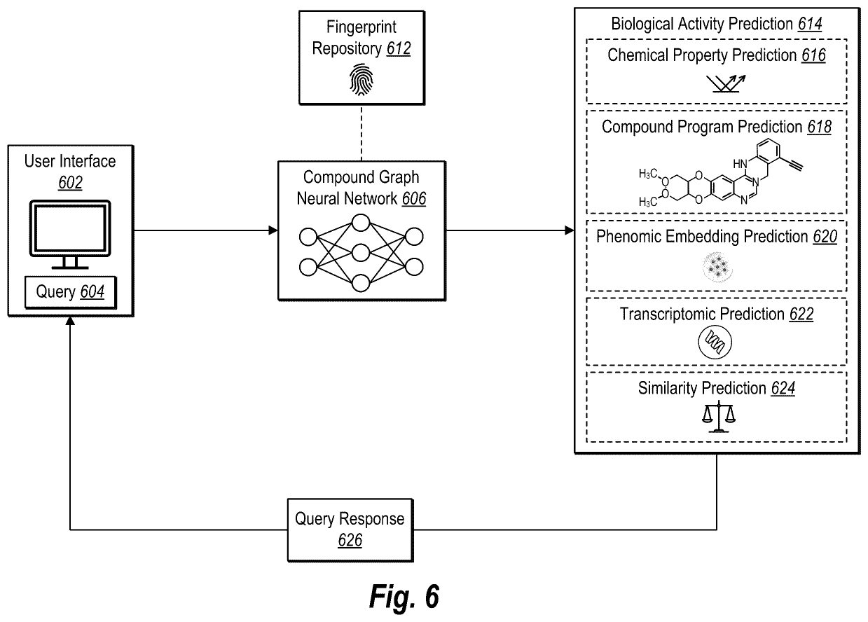

Indeed, as shown in FIG. 2, the molecular graph prediction system utilizes one or more task heads to analyze the post neural network graph representation 220 (e.g., graph-level task heads, task head 224 and task head 226) and the post neural network node representation(s) 222 (e.g., node-level task heads, task head 228 and task head 230). The molecular graph prediction system can implement the task heads in a variety of ways, including as MLPs, as linear layers, as convolutional layers, as recurrent layers, or as attention mechanisms, among others. Specifically, the molecular graph prediction system can utilize the pre-trained prediction heads to analyze the post neural network graph representation 220 and the post neural network node representation(s) 222 and to generate a prediction 234. As will be discussed below in FIG. 6, the molecular graph prediction system can pre-train the task heads for different task predictions relating to the input compound 202.

As shown in FIG. 2., the molecular graph prediction system can utilize the one or more task heads to perform one or more tasks relating to the input compound, such as to generate the prediction 234. Specifically, the molecular graph prediction system can utilize one or more task heads to generate predictions for quantum physics tasks 236 related to the input compound 202. The molecular graph prediction system can generate these predictions at the graph-level or the node-level. For example, the molecular graph prediction system can predict the molecular energy, the molecular properties (e.g., dipole moments, polarizability), the material properties (e.g., band gaps, electronic band structures), quantum mechanical properties (e.g., electron density distributions, molecular orbitals, vibrational frequencies), or quantum phase predictions of the input compound 202. Similarly, the molecular graph prediction system can predict charges of the atoms for node level predictions.

In addition, as illustrated in FIG. 2, the molecular graph prediction system can utilize one or more task heads to generate predictions for chemistry tasks 238 relating to the input compound 202. The molecular graph prediction system can generate these predictions at the graph-level or the node-level. For example, the molecular graph prediction system can predict the solubility or lipophilicity of the input compound 202. The molecular graph prediction system can make chemical reaction predictions (e.g., reaction type, product formation, reaction mechanisms) for the input compound 202. The molecular graph prediction system can predict the electronic structure of the input compound 202.

As illustrated in FIG. 2, the molecular graph prediction system can utilize one or more task heads to generate predictions for biology tasks 240 relating to the input compound 202. The molecular graph prediction system can generate these predictions at the graph-level or the node-level. The molecular graph prediction system can utilize one or more graph-level task heads to generate predictions for the entirety of the input compound. For example, the molecular graph prediction system can predict the toxicity of the input compound 202. The molecular graph prediction system can predict interactions of the input compound 202 with one or more biological targets. The molecular graph prediction system can predict interactions of the input compound 202 with pharmaceutical compounds. The molecular graph prediction system can predict one or more metabolic pathways for the input compound 202.

As shown in FIG. 2, the molecular graph prediction system can perform an act 242 of updating the parameters of the compound graph neural network. For example, the molecular graph prediction system can compare the predictions 234 with ground truth data to update these parameters. To illustrate, the molecular graph prediction system can utilize a loss function to compare the predictions 234 with ground truth data and generate a measure of loss. The molecular graph prediction system can then utilize the measure of loss to modify parameters of the compound graph neural network (e.g., utilizing backpropagation and/or gradient descent). The molecular graph prediction system can update the parameters of various subcomponents of the compound graph neural network, including the encoder manager 216 (and other encoders discussed above for generating encodings), the graph neural network 218, and/or the task heads (e.g., task head 224, task head 226, task head 228, task head 230).

As just described, the molecular graph prediction system can utilize a variety of architectures for the compound graph neural network. For example, in one or more implementations, the molecular graph prediction system utilizes the machine learning architecture as described in Graphium, available at https://graphium-docs.datamol.io/stable/design.html, which is incorporated by reference herein in its entirety. Similarly, in one or more embodiments, the molecular graph prediction system utilizes architectures described by Medez-Lucio, et al. in MolE: A Molecular Foundation Model for Drug Discovery, arXiv.2211.02657, November 2022, which is incorporated by reference herein in its entirety.

Although FIG. 2 depicts an architecture for a compound neural network that the molecular graph prediction system can utilize to analyze an input compound and make various predictions according to the properties of the input compound, the molecular graph prediction system need not be limited to this architecture and can utilize compound graph neural networks having other architectures.

For example, in some embodiments, the molecular graph prediction system can utilize a machine learning model architecture that includes a biased global attention network and a local message passing neural network. The molecular graph prediction system can provide an attention bias matrix and node features as inputs to the biased global attention network. The molecular graph prediction system can provide the node features, edge features, and global features to the local message passing neural network.

For example, the molecular graph prediction system can utilize the biased global attention module to apply a biased attention matrix to a vector representation of node features representative of atoms of an input compound. Specifically, the molecular graph prediction system can utilize the biased global attention network to apply biases to node features representative of atoms in an input compound, including positional bias (e.g., prioritizing atoms based on their positions within the molecular structure of the input compound), functional group bias (e.g., biasing attention towards specific functional groups), element bias (e.g., prioritizing certain atoms over others according to their chemical significance), among others.

As mentioned above, the molecular graph prediction system can provide vector representations of the node features, edge features, and graph features as inputs to the local message passing neural network. The molecular graph prediction system can utilize the local message passing neural network to aggregate information from neighboring nodes within the neural network (e.g., the molecular graph prediction system utilizes the local message passing neural network to contextualize the node and edge representations of the input compound according to neighboring structures within the input compound). The molecular graph prediction system can utilize the local message passing neural network to perform a variety of operations on the vector representations. For example, the molecular graph prediction system can gather and scatter the node features and edge features. The molecular graph prediction system can combine the node features, edge features, and global features utilizing operations such as concatenation.

In addition, the molecular graph prediction system can utilize regularization methods such as dropout to prevent overfitting and improve the overall flexibility of the compound graph neural network. Specifically, the molecular graph prediction system can cause the local message passing neural network to apply node dropout techniques (e.g., randomly setting a fraction of the node feature representations to zero), edge dropout techniques (e.g., effectively removing certain connections between atoms), thereby forcing the compound graph neural network to operate on more sparse data inputs. By utilizing the local message passing neural network, the molecular graph prediction system can generate improved, more contextualized graph representations of the node features and edge features of the input compound.

In some embodiments, the molecular graph prediction system can combine the attention weights from the biased global attention network with the improved node features and edge features. For example, the molecular graph prediction system can utilize a feed forward neural network to concatenate the attention weights with the improved node features and edge features.

By utilizing the biased global attention network local message passing neural network, the molecular graph prediction system can contextualize node and edge components of a graph representation with information about neighboring nodes and edges, thus creating a graph representation of the input compound that can be utilized to model various chemical and biological experiments.

For example, in one or more implementations, the molecular graph prediction system utilizes the machine learning architecture as described in “GPS++: An Optimized Hybrid MPNN/Transformer for Molecular Property prediction,” available at arXiv:2212.02229, December 2022 (hereinafter “GPS++”), which is incorporated by reference herein in its entirety.

In one or more implementations, the molecular graph prediction system performs various modifications to the architecture described above. For example, in one or more implementations, the molecular graph prediction system trains the compound graph neural network to a threshold number of parameters. To illustrate, in some implementations, the molecular graph prediction system builds the compound graph neural network during pre-training to at least 1 billion (or 3 billion) parameters. In some embodiments, this approach provides improved performance for subsequent finetuning for alternative tasks.

Furthermore, as mentioned above, in one or more implementations, the molecular graph prediction system pre-trains the compound graph neural network on a large variety of different tasks so that the model learns a variety of interactions across different feature spaces. For example, in some implementations, the molecular graph prediction system trained the compound graph neural network on a threshold number of tasks (e.g., 100 tasks with 100 task heads or 1000 tasks with 1000 task heads). Indeed, the molecular graph prediction system can simultaneously train and learn on a large volume of different tasks so that the model learns features from a variety of different bio-chemical tasks. Moreover, as discussed above, the molecular graph prediction system can train the compound graph neural network on graph level and node level tasks (e.g., to predict the charge of each atom rather than just the global charge of the molecule). Thus, the molecular graph prediction system can learn both atomic/node level feature spaces and compound/graph level feature spaces.

As described above, the molecular graph prediction system can extract a fingerprint from one or more layers of a pre-trained prediction head and utilize the fingerprint to finetune a compound graph neural network to make a new biological activity prediction for the input compound. In particular, the molecular graph prediction system can utilize fingerprints to finetune and implement compound graph neural networks to generate biological activity predictions that were not part of the initial training. For example, FIG. 3 illustrates the molecular graph prediction system receiving an input compound and utilizing the compound graph neural network of FIG. 2 to generate a new biological activity prediction for the input compound (relative to the predictions 234 of FIG. 2).

As shown in FIG. 3, the molecular graph prediction system receives an input compound 302. The input compound 302 can be the input compound 202 of FIG. 2, the input compound 100 of FIG. 1, or a new compound. As mentioned previously, the molecular graph prediction system can receive the input compound 302 (e.g., from a user input of a query via a client device) and generate a digital representation of the input compound 302.

As illustrated, the molecular graph prediction system can perform the act 204 (featurization, including the act 206 of positional encoding, the act 208 of edge featurization, and the act 210 of node featurization). In addition, similar to the process described in FIG. 2, the molecular graph prediction system can generate a pre-neural network node encoding 312, a pre-neural network edge encoding 314, and feature representations from an encoder manager 316.

As illustrated in FIG. 3 (and similar to the process described above with regard to FIG. 2), the molecular graph prediction system can utilize the graph neural network 218 to analyze the pre-neural network node encodings 312, pre-neural network edge encodings 314, and feature representations of the encoder manager 316 to generate a post neural network node representation(s) 322 and a post neural network graph representation 320. The molecular graph prediction system can structure the post neural network graph representation 320 to include nodes that are representative of atoms of the input compound 302 and edges that are representative of the bonds between atoms of the input compound 302.

As illustrated in FIG. 3, the molecular graph prediction system can utilize one or more pre-trained prediction heads (e.g., the task heads 224, 226, 228, and 230 of FIG. 2) to generate one or more predictions for a first task. As used herein, the term “pre-trained prediction heads” (or pre-trained task heads) refers to task heads that have been trained to generate a particular prediction (or perform a particular task). For example, the molecular graph prediction system trains the task heads 224, 226, 228, and 230 with regard to the process of FIG. 2. Thereafter, the molecular graph prediction system can utilize these task heads as pre-trained task heads 224, 226, 228, and 230 for generating predictions and/or finetuning a compound graph neural network to generate additional predictions utilizing additional task heads. Thus, although FIG. 2 shows the task heads 224-230, FIG. 3 shows pre-trained prediction heads 224-230, because FIG. 3 depicts the molecular graph prediction system fine-tuning the trained compound graph neural network in accordance with one or more embodiments.

Specifically, FIG. 2 illustrates the molecular graph prediction system utilizing a task head (e.g., task head 224) to generate a prediction for a first task (e.g., a prediction 234). The molecular graph prediction system can cause the pre-trained prediction head to generate the prediction 234 from an additional graph representation of an additional input compound. Subsequently, the molecular graph prediction system can modify the parameters of the task head by comparing the prediction for the first task (e.g., the prediction 234) with a ground truth for the first task. By modifying the parameters of the task head 224 subsequent to the comparison of the prediction for the first task with a ground truth for the first task, the molecular graph prediction system trains the task head 224 to become the pre-trained prediction head 224.

As illustrated in FIG. 3., the molecular graph prediction system can utilize the pre-trained prediction head 224, the pre-trained prediction head 226, the pre-trained prediction head 228, and the pre-trained prediction head 230 to make predictions at a graph level. Indeed, the molecular graph prediction system can extract a fingerprint of the input compound 302 generated from one or more internal layers of a pre-trained prediction head.

As used herein, the term “fingerprint” refers to a feature representation from a layer of a machine learning model. For instance, a fingerprint can include a feature vector generated by a layer of a compound graph neural network. In one or more implementations, a fingerprint includes a feature vector generated by a hidden layer of a task head (e.g., a pre-trained prediction head) of a compound graph neural network or a layer of a graph output network. As described above, in some implementations, the molecular graph prediction system utilizes a graph output network to generate a feature vector that is utilized by one or more task heads to generate a prediction. For example, the molecular graph prediction system can utilize a neural network (e.g., an MLP) to process features after being aggregated from the node to the graph level to generate a feature vector for one or more task heads. In some implementations, the molecular graph prediction system utilizes features from the graph output network as a fingerprint. A fingerprint can include a feature representation at a graph level (e.g., from a post neural network graph representation) and/or a feature representation at a node level (e.g., from a post neural network node representation).

For example, in relation to FIG. 3, the fingerprint can be a feature representation (e.g., a feature vector from a layer of a neural network) representing the input compound 302. The molecular graph prediction system can extract the fingerprint by extracting information from the post neural network graph representation 320, one or more post neural network node representations 322, or from the pre-trained prediction head 224, the pre-trained prediction head 226, the pre-trained prediction head 228, or the pre-trained prediction head 230. The molecular graph prediction system can extract the fingerprint (e.g., the fingerprint 332, the fingerprint 334, the fingerprint 336, or another fingerprint) at a graph-level or a node-level. For example, the molecular graph prediction system can extract a fingerprint 332 from the post neural network graph representation 320 and utilize the fingerprint 332 as a high-level representation of the input compound 302. Similarly, the molecular graph prediction system can extract a fingerprint from the post neural network node representation 322.

As shown in FIG. 3, the molecular graph prediction system can extract the fingerprint 334 from the pre-trained prediction head 224 (e.g., a second fingerprint generated from internal layers of a second pre-trained prediction head of the compound graph neural network) based on the post neural network graph representation (e.g., the graph representation). For example, in the instance where the molecular graph prediction system can utilize the pre-trained prediction head 224 to make predictions about the toxicity of the input compound 302, the molecular graph prediction system can utilize the fingerprint 334 as a high-level representation of biological, physical, and/or chemical features relating to toxicity of the input compound 302. The molecular graph prediction system can extract the fingerprint 336 from the pre-trained prediction head 226.

As illustrated in FIG. 3., the molecular graph prediction system can utilize a first neural network (e.g., the MLP 338) to generate a first fingerprint feature representation from the fingerprint 332 based on the graph representation (e.g., the post neural network graph representation 320). The molecular graph prediction system can utilize a second neural network (the MLP 340), to generate a second fingerprint feature representation from the fingerprint 334 based on the graph representation. The fingerprint feature representations can be a feature vector generated from a fingerprint (e.g., by a neural network). In this manner, the molecular graph prediction system can learn to transform fingerprints for a new task.

For example, as shown in FIG. 3, the molecular graph prediction system utilizes the MLP 338 to generate a feature representation from the fingerprint 332. Similarly, the molecular graph prediction system generates a feature representation (e.g., a feature vector) from the fingerprint 334 utilizing the MLP 340 (e.g., a second neural network). Moreover, the molecular graph prediction system generates a feature representation from the fingerprint 334 utilizing the MLP 342. The molecular graph prediction system can then utilize the MLP 344 (e.g., a third neural network) to combine one or more feature representations and make a biological activity prediction 346 (e.g., a prediction for the input compound with regard to a second task).

As discussed above, the biological activity prediction 346 is a new task/prediction compared to the predictions 234. Thus, the molecular graph prediction system can finetune the compound graph neural network utilizing the fingerprints 332-336 to generate a new type of prediction. The learned interactions from the graph neural network 218 and/or the task heads 224-226 are reflected in the fingerprints 332-336. The molecular graph prediction system can utilize MLPs 338-344 to analyze these fingerprints for the biological activity prediction 346. Accordingly, the molecular graph prediction system efficiently finetunes the compound graph neural network. Additional information on biological activity predictions 346 will be discussed below in FIG. 6.

As shown in FIG. 3, the molecular graph prediction system can perform an act 348 of updating parameters of the compound graph neural network. Similar to the act 242, the molecular graph prediction system can compare the biological activity prediction 346 with ground truth data, determine a measure of loss, and modify and update the parameters of the compound graph neural network. However, in one or more implementations, the molecular graph prediction system freezes (e.g., leaves unchanged) some parameters while modifying other parameters. For instance, in one or more implementations, during finetuning the molecular graph prediction system modifies parameters for the MLPs 338-344 based on the measure of loss while freezing other parameters. To illustrate, the molecular graph prediction system can freeze parameters of various encoders described above (e.g. utilized with the act 204 in generating pre-neural network encodings), the graph neural network 218, and the pre-trained prediction heads 224-230. Moreover, the molecular graph prediction system can perform the act 348 to update the parameters of a second neural network (e.g., MLP 340 or MLP 342) and the parameters of the third neural network (e.g., MLP 344) by comparing the biological activity prediction (e.g., a prediction for the input compound 302 with regard to a second task) generated using the second and third neural networks with a ground truth for the input compound 302.

By finetuning the parameters of the pre-trained prediction heads to enable the molecular graph prediction system to make the biological activity predictions 346, the molecular graph prediction system can address problems faced by conventional systems, discussed above, with regard to operational accuracy, inflexibility, and efficiency. Specifically, by extracting fingerprints and generating feature representations from the fingerprints, the molecular graph prediction system can increase operational flexibility by making biological activity predictions 346 that conventional systems cannot, and reduce the computational resources required to accurately generate the biological activity predictions 346 and finetune the compound graph neural network.

Although not illustrated in FIG. 3, the molecular graph prediction system can utilize a compound graph neural network for a variety of additional purposes. For example, the molecular graph prediction system can extract a fingerprint from a post neural network node representation 322. Thus, although not illustrated the molecular graph prediction system can also utilize the fingerprints extracted from the post neural network node representation 322 or the pre-trained prediction heads 228-230 to generate a new type of prediction. (e.g., the molecular graph prediction system can extract node-level fingerprints or can extract fingerprints from node-level pre-trained prediction heads). The learned interactions from the graph neural network 218 and/or the task heads 228-230 are reflected in the node-level pre-trained prediction heads. The molecular graph prediction system can utilize additional neural networks (e.g., additional MLPs) to analyze these fingerprints to make biological activity predictions. Indeed, the molecular graph prediction system can extract a fingerprint from one of the post neural network node representations 322 as a high-level representation of a component of the input compound 302. By extracting one or more node-level fingerprints, the molecular graph prediction system can finetune the compound graph neural network by enabling the compound graph neural network to specifically analyze a unique sub-component of the input compound 302.

As mentioned above, in some embodiments, the molecular graph prediction system includes a graph output neural network (e.g., an MLP) that processes features after being aggregated from the node level (e.g., the post neural network node representation(s) 322) to the graph level (e.g. the post neural network graph representation 320). Additionally, the molecular graph prediction system can utilize the graph output neural network to process these features in preparation for analysis by one or more tasks heads. In one or more implementations, the molecular graph prediction system can extract a fingerprint from one or more layers of the graph output neural network. For example, the molecular graph prediction system can extract the fingerprint from a final layer of the aggregator neural network (or another layer), and provide the fingerprint to a task head (e.g., for finetuning or inference).

Although many of the fingerprints discussed herein refer to fingerprints extracted from various feature vectors/representations of the compound graph neural network, in some embodiments, the molecular graph prediction system can also utilize compound fingerprints generated from other sources (e.g., third-party fingerprints such as RDKit). For example, the molecular graph prediction system can access and utilize compound fingerprints of the input compound (e.g., numerical representations of the structure, atoms, or properties of a compound) that are not generated by a machine learning model or neural network. In some implementations, the molecular graph prediction system can combine fingerprints extracted from the compound graph neural network with these other compound fingerprints. For example, in training and/or implementation, the molecular graph prediction system can concatenate (or otherwise combine) a compound fingerprint with a fingerprint extracted from the compound graph neural network to generate a combined fingerprint and utilize a neural network (e.g., an MLP) to analyze the combined fingerprint and generate a combined fingerprint representation. The molecular graph prediction system can also analyze multiple combined fingerprint representations (e.g., utilizing another neural network) to generate bioactivity predictions. Accordingly, the molecular graph prediction system can utilize a combination of fingerprints extracted from the compound graph neural network and other compound fingerprints to generate the biological activity prediction.

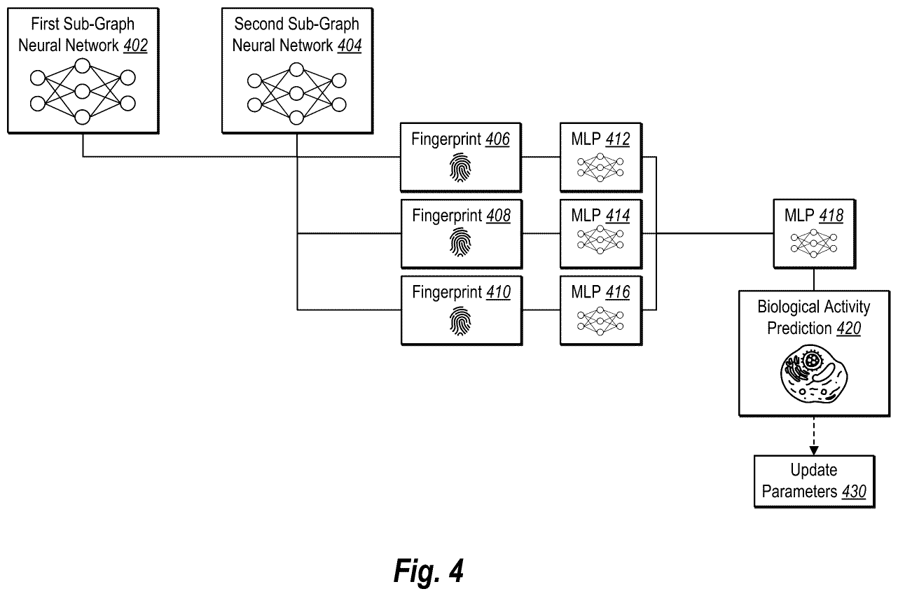

As described above, the molecular graph prediction system can extract a fingerprint from one or more layers of a pre-trained prediction head and utilize the fingerprint to finetune a compound graph neural network to make a new biological activity prediction for the input compound. In particular, the molecular graph prediction system can extract fingerprints from multiple sub-graph neural networks of a compound graph neural network and can utilize the fingerprints to finetune and implement the compound graph neural network to generate biological activity predictions that were not part of the initial training of the compound graph neural network. For example, FIG. 4 illustrates the molecular graph prediction system extracting fingerprints from a first sub-graph neural network and a second sub-graph neural network and utilizing the fingerprints to generate a new biological activity prediction for the input compound (relative to the predictions 234 of FIG. 2).

As shown in FIG. 4, the molecular graph prediction system can utilize a first sub-graph neural network 402 and a second sub-graph neural network 404 to generate a new biological activity prediction 420. More information regarding biological activity predictions will be provided below in FIG. 6.

As used herein, the term “sub-graph neural network” refers to one or more graph neural networks within a compound graph neural network architecture. For example, although FIG. 2 illustrates a single graph neural network architecture, the molecular graph prediction system can train multiple different graph neural networks as illustrated in FIG. 2. The molecular graph prediction system can then combine both of these networks as a new compound graph neural network. In such circumstances, each individual graph neural network is referred to as a sub-graph neural network.

Thus, for example, a sub-graph neural network can include one or more input layers that receive an input compound (e.g., the input compound 100 of FIG. 1, the input compound 202 of FIG. 2, or the input compound 302 of FIG. 3). Additionally, the sub-graph neural network can include one or more layers that featurize the input compound (e.g., the act 204 featurization of FIG. 2 or FIG. 3). Indeed, the sub-graph neural network can include one or more MLP encoders to generate node encodings and/or edge encodings from the input compound. Moreover, the sub-graph neural network can include a transformer architecture to generate node-level representations and graph-level representations of the input compound. Indeed, the sub-graph neural network can include one or more pre-trained prediction heads that can be utilized to extract information from the graph representation.

The molecular graph prediction system can also utilize different sub-graph neural networks having different architectures. For example, in some implementations, the molecular graph prediction system utilizes a first sub-graph neural network having a message passing neural network architecture. Moreover, in some implementations, the molecular graph prediction system utilizes GPS++ as a second sub-graph neural network. In some implementations, the molecular graph prediction system can utilize a different architecture, such as a convolutional, attention mechanism, message passing, or recurrent neural architecture, or a combination/hybrid neural network architecture. Thus, the molecular graph prediction system can combine the learned features from multiple different architectures and multiple different tasks in finetuning a compound graph neural network for new tasks.

As shown in FIG. 4, the molecular graph prediction system utilizes a compound graph neural network having a first sub-graph neural network 402 and a second sub-graph neural network 404. The molecular graph prediction system can pre-train the first sub-graph neural network 402 in a different manner (e.g., using different training data, different task, and/or different architecture/training approaches) than utilized to pre-train the second sub-graph neural network 404. Indeed, the molecular graph prediction system can utilize different architectures of the first sub-graph neural network 402 and the second sub-graph neural network 404 such that the first sub-graph neural network 402 and the second sub-graph neural network generate different graph representations of the same input compound.

For example, the molecular graph prediction system can utilize the first sub-graph neural network 402 to generate a first graph representation of an input compound. The molecular graph prediction system can utilize the second sub-graph neural network 404 to generate a second graph representation of the input compound. Indeed, the molecular graph prediction system can utilize a first set of pre-trained prediction heads to generate a first set of one or more predictions for a first task from the first graph representation. Additionally, the molecular graph prediction system can utilize a second set of pre-trained prediction heads to generate a second set of one or more predictions for a second task from the second graph representation. The molecular graph prediction system can utilize the first set of pre-trained prediction heads and/or the second set of pre-trained prediction heads to make predictions at a graph level.

As illustrated in FIG. 4., the molecular graph prediction system can extract one or more fingerprints from the first sub-graph neural network 402 and/or the second sub-graph neural network 404. For example, the molecular graph prediction system can extract a fingerprint 406 from a pre-trained prediction head of the first set of pre-trained prediction heads (e.g., a pre-trained prediction head of the first sub-graph neural network). Additionally, the molecular graph prediction system can extract a fingerprint 408 from the first graph representation of the input compound (e.g., a graph representation generated from the first sub-graph neural network 402). Moreover, the molecular graph prediction system can extract a fingerprint 410 from a component of the second sub-graph neural network 404, such as from a pre-trained prediction head of the second set of pre-trained prediction heads, or from a second graph representation of the input compound.

Similar to the fingerprints described in FIG. 3 (e.g., the fingerprint 332, the fingerprint 334, and/or the fingerprint 336), the fingerprint 406, the fingerprint 408, and/or the fingerprint 410, can be a feature representation (e.g., a feature vector from a layer or a neural network) representing the input compound. The molecular graph prediction system can extract the fingerprint by extracting information from the first graph representation (e.g., the graph representation generated by the first sub-graph neural network 402). The molecular graph prediction system can extract the fingerprint by extracting information from the second graph representation (e.g., the graph representation generated by the second sub-graph neural network 404). The molecular graph prediction system can extract the fingerprint by extracting information from the first set of pre-trained prediction heads (e.g., pre-trained prediction heads of the first sub-graph neural network 402). The molecular graph prediction system can extract the fingerprint by extracting information from the second set of pre-trained prediction heads (e.g., pre-trained prediction heads of the second sub-graph neural network 404).

For example, the molecular graph prediction system can extract the fingerprint 406 from the first sub-graph neural network 402 and utilize the fingerprint 406 as a high-level representation of toxicity attributes of the input compound. Additionally, the molecular graph prediction system can extract the fingerprint 408 from the second sub-graph neural network 404 and utilize the fingerprint 408 as a high-level representation of absorption attributes of the input compound. Specifically, the molecular graph prediction system can utilize the second sub-graph neural network 404 to generate a second graph representation of the input compound. The molecular graph prediction system can extract a second fingerprint (e.g., fingerprint 408) from the second graph representation of the input compound.

The molecular graph prediction system can generate various feature representations from the fingerprints 406-410. For instance, as shown, the molecular graph prediction system utilizes a neural network (e.g., the MLP 412) to generate a feature representation from the fingerprint 406. In addition, the molecular graph prediction system utilizes a second neural network (e.g., MLP 414) to generate a second fingerprint feature representation from the second fingerprint. Similarly, the molecular graph prediction system can utilize other neural networks (e.g., MLP 416) to generate other fingerprint feature representations.

As shown, the molecular graph prediction system can utilize a third neural network (e.g., MLP 418) to combine fingerprints. For example, the molecular graph prediction system utilizes the MLP 418 to combine the first fingerprint feature representation from the first sub-graph neural network 402 and the second fingerprint feature representation from the second sub-graph neural network 404 to generate the prediction for the second task corresponding to the input compound (e.g., the biological activity prediction 420).

After combining the first fingerprint feature representation and the second fingerprint feature representation, the molecular graph prediction system can modify the parameters of the second neural network (e.g., MLP 414) and the parameters of the third neural network (e.g., MLP 418) by comparing the prediction for the second task (e.g., the biological activity prediction 420) with a ground truth for the input compound with regard to the second task. Indeed, the molecular graph prediction system can modify the parameters of the second neural network (e.g., MLP 414) and the third neural network (e.g., MLP 418) while freezing the parameters of the pre-trained prediction head of the first sub-graph neural network 402 and the second sub-graph neural network 404.

As just discussed, the molecular graph prediction system can use one or more neural networks (e.g., an MLP 412, and MLP 414, or an MLP 416) to generate a feature representation from the fingerprint. The feature representation can be a feature vector generated from a fingerprint (e.g., by a first neural network). In this manner, the molecular graph prediction system can learn to transform the fingerprints for a new task. For example, as shown, the molecular graph prediction system utilizes the MLP 416 to generate a feature representation from the fingerprint 410. Similarly, the molecular graph prediction system generates a feature representation from the fingerprint 408 utilizing MLP 414. Moreover, the molecular graph prediction system generates a feature representation from the fingerprint 406 utilizing the MLP 412. The molecular graph prediction system can utilize a second neural network (e.g., the MLP 418) to combine one or more feature representations and make a biological activity prediction 420.

As discussed above, the biological activity prediction 420 is a new task/prediction compared to the predictions 234. The biological activity prediction 420 can be the biological activity prediction 346 of FIG. 3. Thus, as discussed above, the molecular graph prediction system can finetune the compound graph neural network utilizing the fingerprints 412-416 to generate a new type of prediction. The learned interactions from the first sub-graph neural network 402 and/or the second sub-graph neural network 404 are reflected in the fingerprints 412-416. Accordingly, the molecular graph prediction system efficiently finetunes the compound graph neural network. Additional information on biological activity predictions 420 will be discussed below in FIG. 6.

As shown in FIG. 4., the molecular graph prediction system can perform an act 430 of updating parameters of the compound graph neural network. Similar to the acts 348 and 242, the molecular graph prediction system can compare the biological activity prediction 420 with ground truth data, determine a measure of loss, and modify and update the parameters of the compound graph neural network.

As illustrated in FIG. 4, in one or more implementations, during finetuning (e.g., the act 430 of updating parameters) the molecular graph prediction system modifies parameters for the MLPs 412-416 based on the measure of loss. For example, the molecular graph prediction system can utilize the MLP 412 (e.g., a first neural network of the first sub-graph neural network 402) to generate a first fingerprint feature representation from the fingerprint 406. Additionally, the molecular graph prediction system can utilize the MLP 414 (e.g., a second neural network of the second sub-graph neural network 404) to generate a second fingerprint feature representation from the fingerprint 408. Moreover, the molecular graph prediction system can utilize the MLP 418 (e.g., a third neural network) to combine the first and second fingerprint feature representations to generate a biological activity prediction 420 (e.g., a prediction for a second task corresponding to the input compound). Subsequently, the molecular graph prediction system can compare the biological activity prediction with a ground truth and modify the parameters of the MLP 412 (e.g., the first neural network), the MLP 414 (e.g., the second neural network), or the MLP 418 (e.g., the third neural network) according to the comparison. Additionally, in some embodiments, the molecular graph prediction system can freeze parameters of various encoders as described above while modifying others. For example, when modifying the parameters of the MLP 412, the MLP 414, or the MLP 418 (e.g., the first neural network, the second neural network, or the third neural network) as described previously, the molecular graph prediction system can freeze the other components of the compound graph neural network. Specifically, the molecular graph prediction system can freeze the parameters of the first sub-graph neural network 402, the second sub-graph neural network 404, the pre-trained prediction head, the second pre-trained prediction head, and the first neural network (e.g., MLP 412) while modifying the parameters of the second neural network (e.g., the MLP 414) and the third neural network (e.g., the MLP 418).

By finetuning the parameters of the first set of pre-trained prediction heads and/or the second set of pre-trained prediction heads to enable the molecular graph prediction system to address problems faced by conventional systems discussed above, with regard to operational accuracy, inflexibility, and efficiency. Specifically, by extracting fingerprints and generating feature representations from the fingerprints, the molecular graph prediction system can increase operational flexibility by making biological activity predictions 420 that conventional systems cannot, and reduce the computational resources required to accurately generate the biological activity predictions 420.

Although not illustrated in FIG. 4, the molecular graph prediction system can utilize a compound graph neural network for a variety of additional purposes. For example, the molecular graph prediction system can extract a fingerprint at a node-level (e.g., a node of a graph representation of an input compound). The molecular graph prediction system can extract a node-level fingerprint from a first graph representation generated by a first sub-graph neural network of the compound graph neural network. The molecular graph prediction system can extract a node-level fingerprint from a second graph representation generated by a second sub-graph neural network of the compound graph neural network. Similar to how the molecular graph prediction system can finetune the compound graph neural network the molecular graph prediction system can utilize the node-level fingerprints to generate a new prediction. The molecular graph prediction system can utilize additional neural networks (e.g., additional MLPs) to analyze the node-level fingerprints to make the biological activity prediction 420. Indeed, the molecular graph prediction system can extract a fingerprint from a node of one or more graph representations (e.g., a graph representation generated by the first sub-graph neural network and/or the second sub-graph neural network) as a high-level representation of a component of the input compound.

Additionally, in some embodiments, the molecular graph prediction system can freeze base parameters of the compound graph neural network while adding new parameters to the compound graph neural network. In this manner, the molecular graph prediction system can focus on finetuning various parameters and/or components of the compound graph neural network. Indeed, by freezing base parameters of the compound graph neural network while adding new parameters to the compound graph neural network, the molecular graph prediction system can generate new pre-trained prediction heads. In this manner, the molecular graph prediction system addresses problems faced by traditional systems, mentioned above, of high computational expenses and resources required to train new models, by creating and capturing new hidden representations of input compounds.

As described above, the molecular graph prediction system can extract a fingerprint from one or more internal layers of a pre-trained prediction head. For example, FIG. 5 illustrates the molecular graph prediction system extracting a fingerprint from various internal layers of a pre-trained prediction head in accordance with one or more embodiments.

As shown in FIG. 5 the molecular graph prediction system can extract a fingerprint 522 from one or more internal layers of a pre-trained prediction head 502. The pre-trained prediction head 502 can be a pre-trained prediction head of FIG. 3 (e.g., the pre-trained prediction heads 224-230). The pre-trained prediction head 502 can include a first layer 504, a second layer 506, a third layer 508, a fourth layer 510, a fifth layer 512, or additional internal layers. The internal layers of the pre-trained prediction head 502 are neural network layers or neurons of the pre-trained prediction head 502 that transform features received by the pre-trained prediction head 502 (e.g., such as a post neural network graph representation or a post neural network node representation) into a particular output for which the pre-trained prediction head 502 has been trained. The molecular graph prediction system can utilize each internal layer of the pre-trained prediction head 502 to progressively refine the received features into task-specific representations. The internal layers of the pre-trained prediction head (e.g., the first layer 504, the second layer 506, the third layer 508, the fourth layer 510, the fifth layer 512) can be or include layers such as fully connected (dense) layers, activation layers (e.g., ReLU layers, softmax layers, sigmoid layers, Tanh layers, dropout layers, batch normalization layers, pooling layers, concatenation layers, attention layers, reshape layers, or pairwise interaction layers, among others.

As illustrated in FIG. 5., the molecular graph prediction system can extract a fingerprint 522 from the first layer 504 of the pre-trained prediction head 502, the second layer 506 of the pre-trained prediction head 502, the third layer 508 of the pre-trained prediction head 502, the fourth layer 510 of the pre-trained prediction head 502, and/or the fifth layer 512 of the pre-trained prediction head 502. For example, each of these layers can receive input feature vectors, apply parameters/weights corresponding to the particular layer, and generate a modified/intermediate feature vector that is then passed to the subsequent layer. The molecular graph prediction system can extract one or more fingerprints (e.g., one or more of these feature vectors) from one or more internal layers of the pre-trained prediction head 502, thereby creating one or more representations of specific aspects of the input to the pre-trained prediction head 502 (e.g., such as a post neural network graph representation or a post neural network node representation).

As shown in FIG. 5, the molecular graph prediction system can utilize one or more internal layers of the pre-trained prediction head 502 and/or the fingerprint 522 to make a prediction 514. Indeed, the prediction 514 can be a quantum physics task 516. Specifically, the molecular graph prediction system can utilize the fingerprint 522 to generate graph-level or node-level quantum physics tasks related to an input compound. For example, the molecular graph prediction system can utilize the fingerprint to predict the molecular energy, the molecular properties, the material properties, quantum mechanical properties, or quantum phase predictions of the input compound.

In addition, as shown in FIG. 5., the molecular graph prediction system can utilize the fingerprint 522 to generate predictions for chemistry tasks 518 relating to the input compound. The molecular graph prediction system can make these predictions at the graph-level or the node-level. The molecular graph prediction system can make predictions such as reaction type, product formation, or reaction mechanisms for the input compound.

Indeed, as shown in FIG. 5, the molecular graph prediction system can utilize the fingerprint 522 to make predictions for biology tasks 520 relating to the input compound. The molecular graph prediction system can make these predictions at the graph-level or the node-level. For example, the molecular graph prediction system can predict a toxicity of the input compound, a solubility of the input compound, or one or more metabolic pathways of the input compound, among others.

Although FIG. 5 illustrates multiple types of predictions, it will be appreciated that in one or more implementations, a pre-trained prediction head is trained to generate a single, particular type of prediction. Thus, the dashed lines illustrated in FIG. 5 within the predictions 514 can indicate options of a particular type of prediction for which the molecular graph prediction system has trained the pre-trained prediction head 502. In one or more embodiments, the pre-trained prediction head 502 can generate a particular type of prediction of those illustrated in FIG. 5. As discussed previously, the molecular graph prediction system can train different prediction heads to individually generate different types of predictions. For example, the molecular graph prediction system can utilize a first pre-trained prediction head to make predictions for a quantum physics task. The molecular graph prediction system can utilize a second pre-trained prediction head to make predictions for a chemistry task. Moreover, the molecular graph prediction system can utilize a third pre-trained prediction head to make predictions for a biology task.

Additionally, as illustrated in FIG. 5, the molecular graph prediction system can determine to extract the fingerprint 522 from a layer of a plurality of layers of the pre-trained prediction head (e.g., from the first layer 504, the second layer 506, the third layer 508, the fourth layer 510, the fifth layer 512, or another layer). The molecular graph prediction system can select layers (and/or task heads) for extracting fingerprints based on a variety of factors. For example, the molecular graph prediction system can determine the task head and/or the layer of the plurality of layers according to the input compound and/or a task relating to the input compound. For example, the molecular graph prediction system can receive the input compound and a request to determine the lipid solubility of the input compound. The molecular graph prediction system can analyze the layers of the pre-trained prediction head 502 to determine which layer to extract the fingerprint 522 from in order to accomplish this task. Indeed, the molecular graph prediction system can determine a performance history for each layer of the pre-trained prediction head (e.g., a level of accuracy achieved by each layer of the pre-trained prediction head with regard to various input compounds and/or training tasks), and determine which layer to extract the fingerprint from according to the performance history.

Although not shown in FIG. 5, in some embodiments the molecular graph prediction system can analyze performances of the pre-trained prediction heads of the compound graph neural network. The molecular graph prediction system can determine to extract a fingerprint from a first pre-trained prediction head of a plurality of prediction heads according to the input compound and/or a query relating to the input compound (e.g., according to a prediction the molecular graph prediction system makes for an input compound). For example, the molecular graph prediction system can be tasked with predicting a toxicity level for the input compound. The molecular graph prediction system can determine to extract the fingerprint from the first pre-trained prediction head of the plurality of pre-trained prediction heads according to the task of determining the toxicity level of the input compound. For example, the molecular graph prediction system can determine to extract the fingerprint from the first pre-trained prediction head of the plurality of pre-trained prediction heads because the first pre-trained prediction head of the plurality of pre-trained prediction heads was trained (in comparison to other pre-trained prediction heads of the plurality of pre-trained prediction heads) in toxicity level determination. Indeed, the molecular graph prediction system can determine to extract the fingerprint from the first pre-trained prediction head of the plurality of pre-trained prediction heads according to an analysis of historical performances of the plurality of pre-trained prediction heads regarding to the input compound and/or task.

As described above, the molecular graph prediction system can train and finetune one or more compound graph neural networks to make one or more biological activity predictions for an input compound. In particular, the molecular graph prediction system can store fingerprints that were utilized to finetune the compound graph neural network in a repository. For example, FIG. 6 illustrates the molecular graph prediction system receiving a query for an input compound via a user interface, utilizing a compound graph neural network (e.g., the compound graph neural network of FIG. 3) and a fingerprint repository to generate a new biological activity prediction for the input compound, and providing the biological activity prediction to the user interface as a query response.

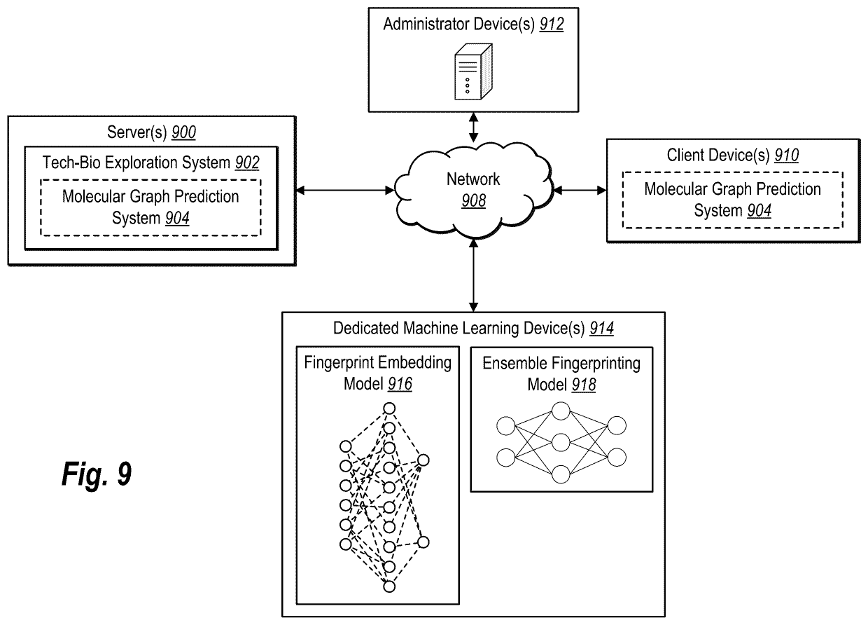

As shown in FIG. 6, the molecular graph prediction system can include a user interface 602. The user interface 602 can be a user interface of a client device, such as a graphical user interface on a cell phone, tablet, or laptop, among others. More information on the client device and its technical environment will be described below in FIG. 9.

As illustrated in FIG. 6., the molecular graph prediction system can receive a query 604 via the user interface 602. The query 604 can be a prompt about an input compound (e.g., the input compound 100 of FIG. 1., the input compound 202 of FIG. 2, or the input compound 302 of FIG. 3). Specifically, the query 604 can be an inquiry relating to a biological activity prediction 614 of the input compound. For example, the molecular graph prediction system can provide a text input element or drop down element and receive (based on user interaction with one or more of these elements) a query 604 about a chemical property prediction 616 for the input compound. The molecular graph prediction system can receive a query 604 for a compound program prediction 618 of the input compound. The molecular graph prediction system can receive a query 604 for a phenomic embedding prediction 620 of the input compound. The molecular graph prediction system can receive a query 604 for a transcriptomic prediction 622 of the input compound. The molecular graph prediction system can receive a query 604 for a similarity prediction 624 of the input compound. In addition, the molecular graph prediction system can receive a query 604 relating to other forms of predictions. For example, the molecular graph prediction system can receive a query relating to a generative task (e.g., generating a new or modified compound).

As illustrated in FIG. 6., the molecular graph prediction system can utilize a compound graph neural network 606 to generate the biological activity prediction 614. Indeed, the molecular graph prediction system can utilize a compound graph neural network as described in FIG. 3 to generate the biological activity prediction 614. Specifically, the molecular graph prediction system can utilize a compound graph neural network that has been fine-tuned to generate the biological activity prediction 614.

The molecular graph prediction system can generate the biological activity prediction 614 by extracting a fingerprint from a graph representation of the input compound. Indeed, the molecular graph prediction system can extract a fingerprint at a graph-level (e.g., from the entire graph representation) or at a node-level (e.g., from one or more sub-components of the graph representation). In addition, the molecular graph prediction system can extract a fingerprint from one or more pre-trained prediction heads of the compound graph neural network 606. Specifically, the molecular graph prediction system can extract one or more fingerprints from one or more internal layers of the one or more pre-trained prediction heads of the compound graph neural network, as discussed above in FIG. 5.

Additionally, the molecular graph prediction system can generate the biological activity prediction 614 by extracting one or more fingerprints from one or more sub-graph neural networks, such as a first sub-graph neural network and a second sub-graph neural network as shown in FIG. 4. Indeed, the molecular graph prediction system can ensemble fingerprints from multiple graph representations of an input compound, from a first set of pre-trained prediction heads (e.g., pre-trained prediction heads of the first sub-graph neural network) and from a second set of pre-trained prediction heads (e.g., pre-trained prediction heads of the second sub-graph neural network) to generate the biological activity prediction 614.

In one or more implementations, the molecular graph prediction system extracts fingerprints by retrieving them from a previously stored repository. For example, during a training process (as described in FIG. 2 and/or FIG. 3), the molecular graph prediction system can generate a variety of fingerprints from various task heads for performing various tasks. The molecular graph prediction system can store these fingerprints for later utilization in generating a new prediction utilizing a different task head.

Indeed, as shown in FIG. 6, during the process of training/finetuning the compound graph neural network 606, the molecular graph prediction system can store extracted fingerprints in the fingerprint repository 612. The fingerprint repository can be a database or other suitable form or memory for storing, retrieving, or modifying fingerprints. The molecular graph prediction system can utilize the fingerprint repository 612 to store, retrieve, and/or modify fingerprints extracted from the compound graph neural network 606. Additionally, the molecular graph prediction system can utilize the fingerprint repository 612 to store, retrieve, and/or modify third-party fingerprints, such as compound fingerprints. The molecular graph prediction system can parse the fingerprint repository 612 according to the query 604 to utilize one or more fingerprints (e.g., fingerprints extracted from the compound graph neural network 606 or third-party fingerprints) to generate the biological activity prediction 614. For example, the molecular graph prediction system can search the fingerprint repository 612 for one or more fingerprints previously generated for a query compound utilizing the compound graph neural network.

As shown in FIG. 6, the molecular graph prediction system can generate a chemical property prediction 616 according to the query 604. For instance, after generating or retrieving fingerprints for a query compound, the molecular graph prediction system can process the fingerprints utilizing a particular/finetuned task head (e.g., using the architecture as shown in FIG. 4) and generate the biological activity prediction.

As shown, the molecular graph prediction system can generate a variety of different biological activity predictions. Specifically, according to the query 604, the molecular graph prediction system can predict physical properties of the input compound, such as its boiling point, melting point, density, solubility, viscosity, surface tension, thermal conductivity, specific head capacity, or electrical conductivity, among others. In addition, according to the query 604, the molecular graph prediction system can predict chemical characteristics of the input compound, such as the acidity, partition coefficient, reaction rate, redox potential, heat of formation, entropy, enthalpy of vaporization, flash point, combustion energy, or chemical stability of the input compound, among others. Moreover, according to the query 604, the molecular graph prediction system can predict biological properties of the input compound, such as the toxicity, bioavailability, half-life, inhibitory concentration, effective concentration, metabolic stability, blood brain barrier permeability, hepatoxicity, or carcinogenicity of the input compound.

Moreover, the molecular graph prediction system can utilize the compound graph neural network to generate binding/matching predictions with proteins including protein-pocket scores between compound-protein pairs. For example, the molecular graph prediction system can train a task head of the compound graph neural network 606 to generate the chemical activity prediction 110 by comparing the chemical activity prediction 110 with a known chemical activity prediction for the input compound (e.g., a ground truth, such as for example the predictions 234 of FIG. 2, observed binding affinity between chemicals and proteins, or other observed chemical activities), and updating the parameters of the compound graph neural network 606 based on the comparison.

As illustrated in FIG. 6, the molecular graph prediction system can make a compound program prediction 618 for the input compound according to the query 604. The molecular graph prediction system can generate the compound program prediction 618 by training a task head based on known program outcomes (e.g., ground truth program behavior of previous compounds within one or more drug development programs).

For instance, the molecular graph prediction system can utilize the outputs of pre-trained program prediction heads to train the compound graph neural network 606 to generate the compound program prediction from a query 604 relating to an input compound. Indeed, the molecular graph prediction system can train the compound graph neural network 606 to utilize the outputs of two or more pre-trained prediction heads to generate the compound program prediction 618. Specifically, the molecular graph prediction system can combine the outputs of two or more pre-trained prediction heads, wherein the pre-trained prediction heads were trained to generate different predictions, respectively, to create the compound program prediction 618. For example, the molecular graph prediction system can utilize a first pre-trained prediction head to generate a prediction for a metabolic pathway of an input compound. The molecular graph prediction system can utilize a second pre-trained prediction head to generate a prediction for a lipid solubility of the input compound. The molecular graph prediction system can extract a first fingerprint of the metabolic pathway prediction and a second prediction of the lipid solubility prediction. The molecular graph prediction system can utilize a first neural network and a second neural network (e.g., a first MLP and a second MLP) to generate a first fingerprint feature representation from the first fingerprint and a second fingerprint feature representation from the second fingerprint. Indeed, the molecular graph prediction system can utilize a third neural network to combine the first and second fingerprint feature representations and generate the compound program prediction 618 from the combined fingerprint feature representations. Thereafter, the molecular graph prediction system can compare the compound program prediction 618 with a known compound program prediction (e.g., a ground truth) and update the parameters of the compound graph neural network 606 based on the comparison.

Once trained, the molecular graph prediction system can utilize compound graph neural network 606 (and the newly trained task head) to generate program predictions for query compounds. To illustrate, the molecular graph prediction system can identify potential compounds related to a target gene for treating a disease. The molecular graph prediction system can then utilize a trained prediction head to analyze compound features and determine a likelihood that the one or more potential compounds can be developed into treatments for the disease.

For example, the molecular graph prediction system can identify an anchor compound or anchor gene from the one or more promising potential compounds and/or genes. Upon determination of the one or more promising potential compounds and/or genes, the molecular graph prediction system can determine a program rating for the anchor compound and/or the anchor gene.

In some embodiments, the molecular graph prediction system can utilize the program rating to initiate an industrial program generation (IPG) process. To illustrate, the molecular graph prediction system can utilize the IPG process to identify various components and/or requirements to develop the anchor compound into an advanced treatment for the disease. Specifically, the molecular graph prediction system can initiate the IPG process to identify information such as statistically strong connections in a biological map to patient-informed phenotypes, Trekseq confirmation (e.g., confirming anchor compound and anchor gene relationships utilizing transcriptomics), Structure-Activity Relationships (SAR) confidence, among others. Moreover, the molecular graph prediction system can utilize the program rating to initiate an industrialized compound generation (ICG) process to apply steps subsequent to the IPG process. For example, the molecular graph prediction system can utilize the ICG process to test the anchor compound with various analytical tests (e.g., SAR screens), or to identify other potential compounds related to the anchor compound for use in the treatment of the disease.

In one or more embodiments, the molecular graph prediction system can utilize a program prediction as part of generating a program rating for initiation compound exploration programs, as described in U.S. patent application Ser. No. 18/521,910, titled “UTILIZING BIOLOGICAL MACHINE LEARNING REPRESENTATIONS AND A LANGUAGE MACHINE LEARNING MODEL FOR INITIATING COMPOUND EXPLORATION PROGRAMS,” which is incorporated by reference herein in its entirety.

As shown in FIG. 6., the molecular graph prediction system can generate a phenomic embedding prediction 620 according to the query 604. Indeed, the molecular graph prediction system can utilize the outputs of a phenomic embedding machine learning model to train the compound graph neural network 606 to generate the phenomic embedding prediction 620 from a query 604 relating to an input compound.

The molecular graph prediction system can develop and/or utilize a phenomic embedding machine learning model that generates phenomic image embeddings from digital images. In particular, the molecular graph prediction system can capture digital images of cells after applying perturbations and developing the perturbed cells. The molecular graph prediction system can then utilize the phenomic embedding machine learning model to map the phenomic digital images to a shared feature space that reflects the perturbations applied to the cells.

To illustrate the phenomic embedding machine learning model can be a masked autoencoder or a classification model trained to generate embeddings from phenomic images. For example, the molecular graph prediction system can utilize a model as described in U.S. patent application Ser. No. 18/545,399, titled “UTILIZING MASKED AUTOENCODER GENERATIVE MODELS TO EXTRACT MICROSCOPY REPRESENTATION AUTOCODER EMBEDDINGS,” or UTILIZING MACHINE LEARNING MODELS TO SYNTHESIZE PERTURBATION DATA TO GENERATE PERTURBATION HEATMAP GRAPHICAL USER INTERFACES, U.S. patent application Ser. No. 18/526,707, which are incorporated by reference herein in their entirety.

The molecular graph prediction system can train a task head of the compound graph neural network 606 to generate perturbation embeddings for compounds (e.g., without having to capture a digital image of a cell perturbed by the compound). For example, the molecular graph prediction system can utilize a finetuning approach (as described above) to train a task head to generate perturbation embeddings. Specifically, the molecular graph prediction system can utilize a new task head to generate predicted embedding from input compound features and then compare the predicted embedding with a previous embedding generated by the phenomic embedding machine learning model (and update the model parameters based on the measure of loss). Alternatively, the molecular graph prediction system can train a task head to generate perturbation predictions and then utilize an internal feature vector of the task head as a perturbation embedding.

Indeed, the molecular graph prediction system can utilize a neural network to combine the outputs of two or more pre-trained prediction heads and generate the phenomic embedding prediction 620. For example, the molecular graph prediction system can utilize a first pre-trained prediction head to generate a prediction for a toxicity level of an input compound. The molecular graph prediction system can utilize a second pre-trained prediction head to generate a prediction for a minimum inhibitory concentration of the input compound. The molecular graph prediction system can extract a first fingerprint of the toxicity prediction and a second fingerprint of the minimum inhibitory concentration. The molecular graph prediction system can utilize a first neural network to generate a first fingerprint feature representation of the first fingerprint. The molecular graph prediction system can utilize a second neural network to generate a second fingerprint feature representation of the second fingerprint. The molecular graph prediction system can utilize a third neural network to combine the first and second fingerprint feature representations and generate the phenomic embedding prediction 620 from the first and second fingerprint feature representations. The molecular graph prediction system can compare the phenomic embedding prediction for the input compound to an output of the pre-trained phenomic embedding machine learning model (e.g., a ground truth) and update the parameters of the compound graph neural network 606 accordingly.

Once trained, the molecular graph prediction system can then utilize the task head of the compound graph neural network 606 to generate perturbation embeddings from an input compound while avoiding the time and resources previously required to perform a perturbation experiment. Indeed, the molecular graph prediction system can utilize the compound graph neural network 606 to analyze input features of the compound and generate the phenomic embedding prediction 620. The molecular graph prediction system could then compare the phenomic embedding prediction to other embeddings (e.g., other gene perturbation embeddings or compound perturbation embeddings) to identify similar/different perturbations.

In addition to generating phenomic embeddings of cells (from query compounds), as illustrated in FIG. 6., the molecular graph prediction system can generate a transcriptomic prediction 622 according to the query 604. Indeed, the molecular graph prediction system can train a task head utilizing transcriptomic profiles resulting from cell perturbation experiments. For example, the molecular graph prediction system can receive a set of data wherein a perturbation (or a set of perturbations) is applied to cells of a well in a plate such that each well is associated with a single perturbation or set of perturbations. From this set of data, the molecular graph prediction system can utilize a gene sequencer to generate a count of mRNA transcripts associated with the cells of each well representing a class of perturbation. For example, the molecular graph prediction system can determine a count of the mRNA transcripts from each well associated with each class of perturbations.

Additionally, the molecular graph prediction system can generate a transcriptomic profile for each perturbation. For example, the molecular graph prediction system can utilize the count of the mRNA transcripts for a particular gene perturbation generate the transcriptomic profile. The molecular graph prediction system can generate a data set including the perturbation experiment (e.g., class of perturbation), and the mRNA count for each gene of interest corresponding to the perturbation experiment. In some implementations, the molecular graph prediction system also generates an embedding of the transcriptomic profiles.

The molecular graph prediction system can utilize these transcriptomic profiles (and/or transcriptomic embeddings) to train the compound graph neural network 606 to generate the transcriptomic prediction 622 from the query 604 relating to an input compound. Indeed, the molecular graph prediction system can train the compound graph neural network 606 to utilize the outputs of two or more pre-trained prediction heads to generate the transcriptomic prediction 622. For example, the molecular graph prediction system can utilize a first pre-trained prediction head to generate a first prediction for a biological reactivity of an input compound (e.g., a prediction about how the input compound might interact with other compounds). The molecular graph prediction system can utilize a second pre-trained prediction head to generate a second prediction for a biological activity mechanism for the input compound (e.g., what biological mechanism the input compound uses to affect its target). The molecular graph prediction system can extract a first fingerprint of the first prediction and a second fingerprint of the second prediction. Thereafter, the molecular graph prediction system can utilize a first neural network to generate a first fingerprint feature representation from the first fingerprint. Additionally, the molecular graph prediction system can utilize a second neural network to generate a second fingerprint feature representation from the second fingerprint. Subsequently, the molecular graph prediction system can utilize a third neural network to combine the first and second fingerprint feature representations and generate the transcriptomic prediction 622. Thereafter, the molecular graph prediction system can compare the transcriptomic prediction 622 with an output of the pre-trained transcriptomics machine learning model (e.g., a ground truth), and modify the parameters of the compound graph neural network 606 based on the comparison.

After training, the molecular graph prediction system can then utilize the compound graph neural network 606 to generate transcriptomic profiles (and/or embeddings) from a query compound (without having to perturb cells or count protein expression data). In particular, the molecular graph prediction system can analyze compound features and generate a transcriptomic profile indicating the predicted protein (e.g., RNA) expression resulting from applying that compound to a cell.

As illustrated in FIG. 6., the molecular graph prediction system can generate a similarity prediction 624 according to the query 604. Indeed, the molecular graph prediction system can generate the similarity prediction 624 by comparing one or more of the chemical property prediction 616, compound program prediction 618, phenomic embedding prediction 620, or transcriptomic prediction 622 for the input compound. Indeed, by comparing the various prediction types for the input compound, the molecular graph prediction system can determine correlations between the various prediction types. For example, the molecular graph prediction system could use the similarity prediction 624 to determine what effects a functional group of the input compound (e.g., a node of the graph representation) might have on the chemical property prediction 616, the compound program prediction 618, the phenomic embedding prediction 620, or the transcriptomic prediction 622. Specifically, the molecular graph prediction system can generate the similarity prediction 624 by comparing hidden layers of the various prediction types (e.g., if the chemical property prediction 616 and the compound program prediction 618 are the final layers of their respective parts of the compound graph neural network 606, the molecular graph prediction system can generate the similarity prediction 624 by comparing hidden layers from those components of the compound graph neural network 606). By determining what effects a functional group or an input can have on the various prediction types, the molecular graph prediction system enables implementing systems to create more accurate, flexible, efficient predictions for the behavior of input compounds.

As shown in FIG. 6, the molecular graph prediction system can provide the biological activity prediction 614 to the user interface 602 as the query response 626. The molecular graph prediction system can structure the query response 626 to include several components. For example, the molecular graph prediction system can structure the query response 626 to include structural and/or chemical information about the input compound, (e.g., information regarding the chemical property prediction 616), such as an ADMET prediction for the input compound. Indeed, the molecular graph prediction system can structure the query response 626 to include information about the compound program prediction 618, such as a likelihood of success for development of the input compound. Additionally, the molecular graph prediction system can structure the query response 626 to include information about the phenomic embedding prediction 620, such as an effect of a perturbation on a morphology of a target cell. Moreover, the molecular graph prediction system can structure the query response 626 to include information about the transcriptomic prediction, such as an effect caused by the input compound on mRNA counts of a target gene. In addition, in some embodiments, the molecular graph prediction system can structure the query response 626 to include information from multiple predictions (e.g., one or more of the chemical property prediction 616, the compound program prediction 618, the phenomic embedding prediction 620, the transcriptomic prediction 622, or the similarity prediction 624).

In addition, while not illustrated in FIG. 6, in some embodiments, the molecular graph prediction system can generate a new/modified compound in response to a query. Indeed, the molecular graph prediction system can utilize the compound graph neural network as part of a generative model that creates new or modified compounds. Indeed, the compound graph neural network can be implemented as part of a larger graph neural network that learns the structural properties of molecules for generating new compounds. The compound graph neural network can also be used as part of an oracle for guiding a generative model (e.g., a generative adversarial neural network, variational autoencoder, reinforcement learning model, autoregressive model, transformer such as large language model, diffusion model, or flow based model). For example, the compound graph neural network can act as an oracle to evaluate generated compounds (e.g., chemical properties, biological interactions) and provide feedback to guide the model in generating new or novel compounds.

As mentioned above, the molecular graph prediction system can increase the accuracy, efficiency, and operational flexibility of implementing systems. FIG. 7 depicts a graphical representation of experimental results achieved by an experimental implementation of the molecular graph prediction system. In particular, FIG. 7 illustrates normalized performance on the TDC ADMET Benchmark relative to a variety of different models, including variations in performance as the number of learned parameters increases for an example implementation of the molecular graph prediction system.

Specifically, FIG. 7 depicts the results of utilizing an experimental fingerprinting model (labeled GPS++ in the figure) and an ensemble probing model to train a compound graph neural network to complete TDC ADMET benchmark tasks. The TDC ADMET benchmark is a standardized set of tasks designed to assess the ability of machine learning models to predict important properties of molecules in drug discovery, such as absorption, distribution, metabolism, excretion, and toxicity.

The figure displays how the fingerprint model (e.g., GPS++ in the figure) and ensemble fingerprinting model (e.g., ensemble probing in the figure) performed on TDC ADMET benchmark tasks when each model had been scaled to 1 billion parameters (e.g., the figure displays how increasing the training parameters of a model affects its performance on TDC ADMET benchmark tasks). Indeed, for the fingerprinting model, the molecular graph prediction system extracted multiple fingerprints from different layers of the fingerprinting model and utilized the fingerprints to complete the TDC ADMET benchmark tasks. Additionally, for the ensemble fingerprinting model, the molecular graph prediction system extracted fingerprints from multiple pre-trained models. As depicted in FIG. 7, both the fingerprinting model and the ensemble probing model outperform other models, including TDC Baseline (the normalized score across the best models per task reported in Huang, K., Fu, T., Gao, W., Zhao, Y., Roohani, Y., Leskovec, J., Coley, C. W., Xiao, C., Sun, J., and Zitnik, M. Therapeutics data commons: Machine learning datasets and tasks for drug discovery and development. Proceedings of Neural Information Processing Systems, NeurIPS Datasets and Benchmarks, 2021) and Mendez-Lucio, O., Nicolaou, C., and Earnshaw, B. Mole: A molecular foundation model for drug discovery. arXiv preprint arXiv:2211.02657, 2022.

Indeed, the ensemble probing model almost reaches TDC SOTA performance, which is a remarkable because the SOTA performance score is derived from the best scoring method per task of the benchmark collection. In other words, utilizing the ensemble probing method alone showed nearly equivalent performance to selecting the best scoring method for each individual task.

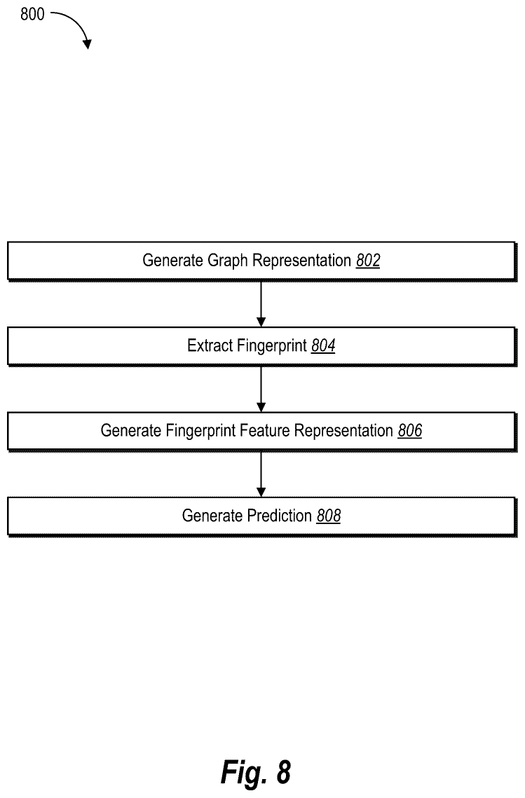

FIGS. 1-7, the corresponding text, and the examples provide a number of different systems, methods, and non-transitory computer readable media for extracting fingerprints from a graph representation of an input compound and utilizing the fingerprints to finetune a compound graph neural network, thus enabling the compound graph neural network to generate biological activity predictions for the input compound. In addition to the foregoing, embodiments can also be described in terms of flowcharts comprising acts for accomplishing a particular result. For example, FIG. 8 illustrates a flowchart of an example sequence of acts in accordance with one or more embodiments.

While FIG. 8 illustrates acts according to some embodiments, alternative embodiments may omit, reorder, and/or modify the acts shown in FIG. 8. The acts of FIG. 8 can be performed as part of a method (e.g., a computer-implemented method). Alternatively, a non-transitory computer readable medium can comprise instructions, that when executed by one or more processors (e.g., at least one processor), cause a computing device to perform the acts of FIG. 8. In still further embodiments, a system can perform the acts of FIG. 8. Additionally, the acts described herein may be repeated or performed in parallel with one another or in parallel with different instances of the same or other similar acts.

FIG. 8 illustrates an example series of acts 800 for generating a biological activity prediction in accordance with one or more embodiments. The series of acts can include acts 802-808 of generating a graph representation of an input compound; extracting a fingerprint of the input compound; generating a first fingerprint feature representation from the fingerprint; and combining the first fingerprint feature representation and a second fingerprint feature representation to generate a prediction for the input compound.

For example, in one or more embodiments, acts 802-808 include generating a graph representation reflecting node features and edge features from an input compound; extracting a fingerprint of the input compound generated from internal layers of a pre-trained prediction head of a compound graph neural network based on the graph representation of the input compound, wherein the pre-trained prediction head is trained to generate predictions for a first task; generating, utilizing a neural network, a first fingerprint feature representation from the fingerprint; or combining the first fingerprint feature representation and a second fingerprint feature representation to generate a prediction for the input compound with regard to a second task.

In one or more implementations, the series of acts 800 include extracting a second fingerprint generated from internal layers of a second pre-trained prediction head of the compound graph neural network based on the graph representation of the input compound, wherein the second pre-trained prediction head is trained to generate predictions for the second task; and generating, by a second neural network, the second fingerprint representation from the second fingerprint.

In addition, in one or more implementations, the series of acts 800 includes combining, utilizing a third neural network, the first fingerprint feature representation and the second fingerprint feature representation to generate the prediction for the input compound with regard to the second task; and modifying parameters of the second neural network and the third neural network by comparing the prediction for the input compound with regard to the second task to a ground truth for the input compound with regard to the second task.

Further, in some implementations, the compound graph neural network includes a graph-level pre-trained prediction head and a node-level pre-trained prediction head, and the series of acts 800 includes extracting the second fingerprint by extracting a graph-level fingerprint from the graph-level pre-trained prediction head of the compound graph neural network.

In one or more implementations, the compound graph neural network comprises a first sub-graph neural network and a second sub-graph neural network, and the first sub-graph neural network comprises the pre-trained prediction head, and the series of acts 800 includes extracting a second fingerprint generated by a second pre-trained prediction head of the second sub-graph neural network based on a second graph representation of the input compound; and generating, utilizing a second neural network, the second fingerprint feature representation from the second fingerprint.

In addition, in some implementations, the series of acts 800 includes combining, utilizing a third neural network, the first fingerprint feature representation from the first sub-graph neural network and the second fingerprint feature representation from the second sub-graph neural network to generate the prediction for the second task corresponding to the input compound.

Further, in one or more implementations, the series of acts 800 includes modifying parameters of the second neural network and a third neural network by comparing the prediction for the input compound with regard to the second task with a ground truth for the input compound with regard to the second task.

In addition, in one or more implementations, the series of acts 800 includes modifying the parameters of the second neural network and the third neural network while freezing parameters the pre-trained prediction head and the compound graph neural network.

Further, in some implementations, the series of acts 800 includes training the pre-trained prediction head of the compound graph neural network by generating, utilizing the pre-trained prediction head, a prediction for the first task from an additional graph representation of an additional input compound; and modifying the parameters of a prediction head by comparing the prediction for the first task with a ground truth for the first task.

Additional detail regarding the molecular graph prediction system environment will now be provided with reference to FIG. 9. In particular, FIG. 9 illustrates a schematic diagram of a system environment in which the molecular graph prediction system can operate in accordance with one or more embodiments.

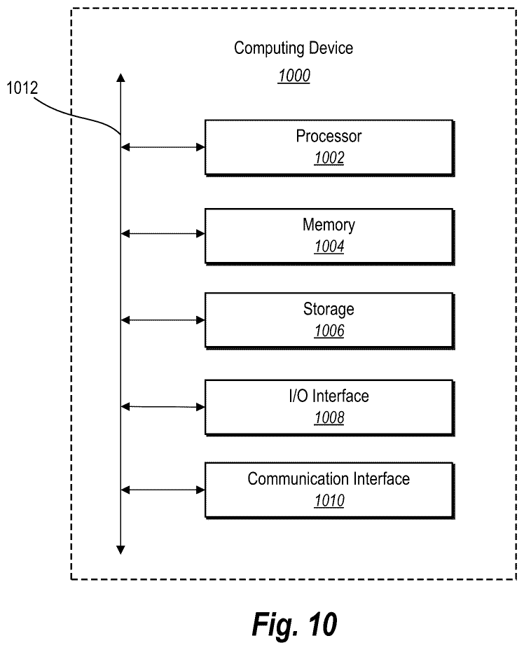

As shown in FIG. 9, the environment includes server(s) 900 (which includes a tech-bio exploration system 902 and the molecular graph prediction system 904), dedicated machine learning device(s) 914, a network 908, client device(s) 910 and administrator device(s) 912. As further illustrated in FIG. 9, the various computing devices within the environment can communicate via the network 908. Although FIG. 9 illustrates the molecular graph prediction system 904 (e.g., the molecular graph prediction system discussed above with regards to FIGS. 1-8) being implemented by a particular component and/or device within the environment, the molecular graph prediction system 904 can be implemented, in whole or in part, by other computing devices and/or components in the environment (e.g., the additional device(s)). Additional description regarding the illustrated computing devices is provided with respect to FIG. 10 below.

As shown in FIG. 9, the server(s) 900 (e.g., one or more local servers operated by a particular entity) can include the tech-bio exploration system 902. In some embodiments, the tech-bio exploration system 902 can determine, store, generate, and/or display tech-bio information including maps of biology, experiments from various sources, and/or machine learning tech-bio predictions. For instance, the tech-bio exploration system 902 can analyze data signals corresponding to various treatments or interventions (e.g., compounds or biologics) and the corresponding relationships in genetics, proteomics, phenomics (i.e., cellular phenotypes), and invivomics (e.g., expressions or results within a living animal). Moreover, the tech-bio exploration system 902 provides an environment for operating, executing, and managing complex drug discovery pipelines.

For instance, the tech-bio exploration system 902 can generate and access experimental results corresponding to gene sequences, protein shapes/folding, protein/compound interactions, phenotypes resulting from various interventions or perturbations (e.g., gene knockout sequences or compound treatments), and/or in vivo experimentation on various treatments in living animals. By analyzing these signals (e.g., utilizing various machine learning models), the tech-bio exploration system 902 can generate or determine a variety of predictions and inter-relationships for improving treatments/interventions.

To illustrate, the tech-bio exploration system 902 can generate maps of biology indicating biological inter-relationships or similarities between these various input signals to discover potential new treatments as part of the complex compound discovery process. For example, the tech-bio exploration system 902 can utilize machine learning and/or maps of biology to identify a similarity between a first gene associated with disease treatment and a second gene previously unassociated with the disease based on a similarity in resulting phenotypes from gene knockout experiments. The tech-bio exploration system 902 can then identify new treatments based on the gene similarity (e.g., by targeting compounds the impact the second gene). Similarly, the tech-bio exploration system 902 can analyze signals from a variety of sources (e.g., protein interactions, or in vivo experiments) to predict efficacious treatments based on various levels of biological data.

The tech-bio exploration system 902 can generate GUIs comprising dynamic user interface elements to convey tech-bio information and receive user input for intelligently exploring tech-bio information. Indeed, as mentioned above, the tech-bio exploration system 902 can generate GUIs displaying different maps of biology that intuitively and efficiently express complex interactions between different biological systems for identifying improved treatment solutions. Furthermore, the tech-bio exploration system 902 can also electronically communicate tech-bio information between various computing devices.

As shown in FIG. 9, the tech-bio exploration system 902 can include a system that facilitates various models or algorithms for generating maps of biology (e.g., maps or visualizations illustrating similarities or relationships between genes, proteins, diseases, compounds, and/or treatments) and discovering new treatment options over one or more networks. For example, the tech-bio exploration system 902 collects, manages, and transmits data across a variety of different entities, accounts, and devices. In some cases, the tech-bio exploration system 902 is a network system that facilitates access to (and analysis of) tech-bio information within a centralized operating system. Indeed, the tech-bio exploration system 902 can link data from different network-based research institutions to generate and analyze maps of biology.

As shown in FIG. 9, the tech-bio exploration system 902 can include a system that comprises the molecular graph prediction system 904 that generates, stores, manages, transmits data pertaining to the generation of graph representations of an input compound and the utilization of that graph representation to generate biological activity predictions for the input compound. For example, in context of the above description for the tech-bio exploration system 902, in some embodiments the tech-bio exploration system 902 further utilizes the molecular graph prediction system 904 to enhance the coordination between various groups involved in the drug discovery process. For instance, the molecular graph prediction system 904 works in tandem with the tech-bio exploration system 902 to extract fingerprints from graph representations, utilize the fingerprints to generate biological activity predictions, transmit the biological activity predictions to one or more devices, and initiate one or more downstream model predictions or processes.

As also illustrated in FIG. 9, the environment includes the client device(s) 910. As mentioned above, the client device(s) 910 can be involved in the process of drug discovery. Thus, for example, the client device(s) 910 can coordinate/manage a first stage of generating a graph representation of an input compound. Moreover, the client device(s) 910 can coordinate/manage a second stage such as extracting a fingerprint from the graph representation. Further, the client device(s) 910 can coordinate/manage a third stage of utilizing the fingerprint to generate a biological prediction to generate one or more additional predictions or initiate one or more programs (IPG or ICG).

To illustrate, the client device(s) 910 can include computing devices that implement or manage a compound program generation stage of a compound discovery process. Similarly, the client device(s) 910 can include computing devices that implement or manage a compound lead generation stage and the client device(s) 910 can include computing devices that implement or manage a compound/dose selection stage. For example, the molecular graph prediction system 904 can receive one or more requests to utilize the dedicated machine learning device(s) 914 to extract one or more fingerprints from a graph representation of an input compound. For instance, the molecular graph prediction system 904 can receive additional requests from the client device(s) 910 that include generating the biological activity predictions.

In some embodiments, the environment also includes additional device(s). For example, the molecular graph prediction system 904 can utilize the additional device(s) to further operate and manage the completion of complex drug discovery pipelines. For instance, the additional device(s) include experimental device(s) and analytical device(s). Further, in some instances, the additional device(s) also include the computing devices discussed below in FIG. 10.

Furthermore, in one or more implementations, the client device(s) 910 include a client application. The client application can include instructions that (upon execution) cause the client device(s) 910 to perform various actions. For example, a user of a user account can interact with the client application on the client device(s) 910 to execute experiments or other multi-faceted processes and to further access tech-bio information, initiate a request for a graph representation, a fingerprint extraction, or a biological activity prediction. For instance, in some embodiments the molecular graph prediction system 904 receives a request to generate a graph representation of an input compound, and in response generates the graph representation and returns the graph representation to the client device(s) 910. In some instances, the transmittal of the graph representation to the client device(s) 910 causes the client device(s) 910 to execute an action (e.g., extract a fingerprint or generate a downstream model prediction).

As shown, the environment can also include dedicated machine learning device(s) 914. For example, the dedicated machine learning device(s) 914 can include computing devices or virtual machines dedicated to training or implementing large-scale machine learning models. For example, the dedicated machine learning device(s) 914 can generate machine learning predictions and/or embeddings based on digital biological data (e.g., digital images of phenotypes resulting from different perturbations or compound-protein interactions from compound features). As shown, the dedicated machine learning device(s) 914 include a fingerprint embedding model 916 and an ensemble fingerprinting model 918. Thus, the molecular graph prediction system 904 interacts with the dedicated machine learning device(s) 914 to extract fingerprints from graph representations of input compounds and generate biological activity predictions for the input compounds utilizing the fingerprints.

The environment can also include experimental device(s). For example, the tech-bio exploration system 902 can interact with the experimental device(s) that include intelligent robotic devices and camera devices for generating and capturing digital images of cellular phenotypes resulting from different perturbations (e.g., genetic knockouts or compound treatments of stem cells). Similarly, the experimental device(s) can include camera devices and/or other sensors (e.g., heat or motion sensors) capturing real-time information from animals as part of in vivo experimentation. The tech-bio exploration system 902 can also interact with a variety of other experimental device(s) such as devices for determining, generating, or extracting gene sequences or protein information. For example, the experimental device(s) may include computing devices linked to biosensors electrophysiological platforms, x-ray crystallography machines, liquid chromatography mass spectrometry systems, nuclear magnetic resonance spectrometers, mass spectrometers. In some implementations, the molecular graph prediction system 904 generates the graph representation, extracts a fingerprint of the graph representation, and further determines to employ or utilize one or more experimental devices (e.g., to initiate one or more experiments based on the graph representations or the fingerprints of the graph representations).

As further shown in FIG. 9, the environment includes the network 908. As mentioned above, the network 908 can enable communication between components of the environment. In one or more embodiments, the network 908 may include a suitable network and may communicate using a various number of communication platforms and technologies suitable for transmitting data and/or communication signals, examples of which are described with reference to FIG. 10. Furthermore, although FIG. 9 illustrates computing devices communicating via the network 908, the various components of the environment can communicate and/or interact via other methods (e.g., communicate directly).

Embodiments of the present disclosure may comprise or utilize a special purpose or general-purpose computer including computer hardware, such as, for example, one or more processors and system memory, as discussed in greater detail below. Embodiments within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., memory), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a “NIC”), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed by a processor, cause a general-purpose computer, special purpose computer, or special purpose processing device to perform a certain function or group of functions. In some embodiments, computer-executable instructions are executed by a general-purpose computer to turn the general-purpose computer into a special purpose computer implementing elements of the disclosure. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Embodiments of the present disclosure can also be implemented in cloud computing environments. As used herein, the term “cloud computing” refers to a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), Platform as a Service (“PaaS”), and Infrastructure as a Service (“IaaS”). A cloud-computing model can also be deployed using different deployment models such as private cloud, community cloud, public cloud, hybrid cloud, and so forth. In addition, as used herein, the term “cloud-computing environment” refers to an environment in which cloud computing is employed.

FIG. 10 illustrates a block diagram of an example computing device 1000 that may be configured to perform one or more of the processes described above. One will appreciate that one or more computing devices, such as the computing device 1000 may represent the computing devices described above. In one or more embodiments, the computing device 1000 may be a mobile device (e.g., a mobile telephone, a smartphone, a PDA, a tablet, a laptop, a camera, a tracker, a watch, a wearable device, etc.). In some embodiments, the computing device 1000 may be a non-mobile device (e.g., a desktop computer or another type of client device). Further, the computing device 1000 may be a server device that includes cloud-based processing and storage capabilities.

As shown in FIG. 10, the computing device 1000 can include one or more processor(s) 1002, memory 1004, a storage device 1006, input/output interfaces 1008 (or “I/O interfaces 1008”), and a communication interface 1010, which may be communicatively coupled by way of a communication infrastructure (e.g., bus 1012). While the computing device 1000 is shown in FIG. 10, the components illustrated in FIG. 10 are not intended to be limiting. Additional or alternative components may be used in other embodiments. Furthermore, in certain embodiments, the computing device 1000 includes fewer components than those shown in FIG. 10. Components of the computing device 1000 shown in FIG. 10 will now be described in additional detail.

In particular embodiments, the processor(s) 1002 includes hardware for executing instructions, such as those making up a computer program. As an example, and not by way of limitation, to execute instructions, the processor(s) 1002 may retrieve (or fetch) the instructions from an internal register, an internal cache, memory 1004, or a storage device 1006 and decode and execute them.

The computing device 1000 includes memory 1004, which is coupled to the processor(s) 1002. The memory 1004 may be used for storing data, metadata, and programs for execution by the processor(s). The memory 1004 may include one or more of volatile and non-volatile memories, such as Random-Access Memory (“RAM”), Read-Only Memory (“ROM”), a solid-state disk (“SSD”), Flash, Phase Change Memory (“PCM”), or other types of data storage. The memory 1004 may be internal or distributed memory.

The computing device 1000 includes a storage device 1006 includes storage for storing data or instructions. As an example, and not by way of limitation, the storage device 1006 can include a non-transitory storage medium described above. The storage device 1006 may include a hard disk drive (HDD), flash memory, a Universal Serial Bus (USB) drive or a combination these or other storage devices.

As shown, the computing device 1000 includes one or more I/O interfaces 1008, which are provided to allow a user to provide input to (such as user strokes), receive output from, and otherwise transfer data to and from the computing device 1000. These I/O interfaces 1008 may include a mouse, keypad or a keyboard, a touch screen, camera, optical scanner, network interface, modem, other known I/O devices or a combination of such I/O interfaces 1008. The touch screen may be activated with a stylus or a finger.

The I/O interfaces 1008 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O interfaces 1008 are configured to provide graphical data to a display for presentation to a user. The graphical data may be representative of one or more graphical user interfaces and/or any other graphical content as may serve a particular implementation.

The computing device 1000 can further include a communication interface 1010. The communication interface 1010 can include hardware, software, or both. The communication interface 1010 provides one or more interfaces for communication (such as, for example, packet-based communication) between the computing device and one or more other computing devices or one or more networks. As an example, and not by way of limitation, communication interface 1010 may include a network interface controller (NIC) or network adapter for communicating with an Ethernet or other wire-based network or a wireless NIC (WNIC) or wireless adapter for communicating with a wireless network, such as a WI-FI. The computing device 1000 can further include a bus 1012. The bus 1012 can include hardware, software, or both that connects components of computing device 1000 to each other.

In one or more implementations, various computing devices can communicate over a computer network. This disclosure contemplates any suitable network. As an example, and not by way of limitation, one or more portions of a network may include an ad hoc network, an intranet, an extranet, a virtual private network (“VPN”), a local area network (“LAN”), a wireless LAN (“WLAN”), a wide area network (“WAN”), a wireless WAN (“WWAN”), a metropolitan area network (“MAN”), a portion of the Internet, a portion of the Public Switched Telephone Network (“PSTN”), a cellular telephone network, or a combination of two or more of these.

In particular embodiments, the computing device 1000 can include a client device that includes a requester application or a web browser, such as MICROSOFT INTERNET EXPLORER, GOOGLE CHROME, or MOZILLA FIREFOX, and may have one or more add-ons, plug-ins, or other extensions, such as TOOLBAR or YAHOO TOOLBAR. A user at the client device may enter a Uniform Resource Locator (“URL”) or other address directing the web browser to a particular server (such as server), and the web browser may generate a Hyper Text Transfer Protocol (“HTTP”) request and communicate the HTTP request to server. The server may accept the HTTP request and communicate to the client device one or more Hyper Text Markup Language (“HTML”) files responsive to the HTTP request. The client device may render a webpage based on the HTML files from the server for presentation to the user. This disclosure contemplates any suitable webpage files. As an example, and not by way of limitation, webpages may render from HTML files, Extensible Hyper Text Markup Language (“XHTML”) files, or Extensible Markup Language (“XML”) files, according to particular needs. Such pages may also execute scripts such as, for example and without limitation, those written in JAVASCRIPT, JAVA, MICROSOFT SILVERLIGHT, combinations of markup language and scripts such as AJAX (Asynchronous JAVASCRIPT and XML), and the like. Herein, reference to a webpage encompasses one or more corresponding webpage files (which a browser may use to render the webpage) and vice versa, where appropriate.

In particular embodiments, the tech-bio exploration system 902 may include a variety of servers, sub-systems, programs, modules, logs, and data stores. In particular embodiments, the tech-bio exploration system 902 may include one or more of the following: a web server, action logger, API-request server, transaction engine, cross-institution network interface manager, notification controller, action log, third-party-content-object-exposure log, inference module, authorization/privacy server, search module, user-interface module, user-profile (e.g., provider profile or requester profile) store, connection store, third-party content store, or location store. The tech-bio exploration system 902 may also include suitable components such as network interfaces, security mechanisms, load balancers, failover servers, management-and-network-operations consoles, other suitable components, or any suitable combination thereof. In particular embodiments, the tech-bio exploration system 902 may include one or more user-profile stores for storing user profiles and/or account information for credit accounts, secured accounts, secondary accounts, and other affiliated financial networking system accounts. A user profile may include, for example, biographic information, demographic information, financial information, behavioral information, social information, or other types of descriptive information, such as interests, affinities, or location.

The web server may include a mail server or other messaging functionality for receiving and routing messages between the tech-bio exploration system 902 and one or more client devices. An action logger may be used to receive communications from a web server about a user's actions on or off the tech-bio exploration system 902. In conjunction with the action log, a third party-content-object log may be maintained of user exposures to third party-content objects. A notification controller may provide information regarding content objects to a client device. Information may be pushed to a client device as notifications, or information may be pulled from a client device responsive to a request received from the client device. Authorization servers may be used to enforce one or more privacy settings of the users of the tech-bio exploration system 902. A privacy setting of a user determines how particular information associated with a user can be shared. The authorization server may allow users to opt in to or opt out of having their actions logged by the tech-bio exploration system 902 or shared with other systems, such as, for example, by setting appropriate privacy settings. Third party-content-object stores may be used to store content objects received from third parties. Location stores may be used for storing location information received from a client device associated with users.

In the foregoing specification, the invention has been described with reference to specific example embodiments thereof. Various embodiments and aspects of the invention(s) are described with reference to details discussed herein, and the accompanying drawings illustrate the various embodiments. The description above and drawings are illustrative of the invention and are not to be construed as limiting the invention. Numerous specific details are described to provide a thorough understanding of various embodiments of the present invention.

The present invention may be embodied in other specific forms without departing from its spirit or essential characteristics. The described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel to one another or in parallel to different instances of the same or similar steps/acts. The scope of the invention is, therefore, indicated by the appended claims rather than by the foregoing description. All changes that come within the meaning and range of equivalency of the claims are to be embraced within their scope.

What is claimed is:

1. A computer-implemented method comprising:
generating a graph representation reflecting node features and edge features from an input compound;
extracting a fingerprint of the input compound generated from internal layers of a pre-trained prediction head of a compound graph neural network based on the graph representation of the input compound, wherein the pre-trained prediction head is trained to generate predictions for a first task;
generating, utilizing a neural network, a first fingerprint feature representation from the fingerprint; and
combining the first fingerprint feature representation and a second fingerprint feature representation to generate a prediction for the input compound with regard to a second task.

2. The computer-implemented method of claim 1, further comprising:
extracting a second fingerprint generated from internal layers of a second pre-trained prediction head of the compound graph neural network based on the graph representation of the input compound, wherein the second pre-trained prediction head is trained to generate predictions for the second task; and
generating, by a second neural network, the second fingerprint feature representation from the second fingerprint.

3. The computer-implemented method of claim 2, further comprising:
combining, utilizing a third neural network, the first fingerprint feature representation and the second fingerprint feature representation to generate the prediction for the input compound with regard to the second task; and
modifying parameters of the second neural network and the third neural network by comparing the prediction for the input compound with regard to the second task to a ground truth for the input compound with regard to the second task.

4. The computer-implemented method of claim 2, wherein the compound graph neural network comprises a graph-level pre-trained prediction head and a node-level pre-trained prediction head, and extracting the second fingerprint comprises extracting a graph-level fingerprint from the graph-level pre-trained prediction head of the compound graph neural network.

5. The computer-implemented method of claim 1, wherein the compound graph neural network comprises a first sub-graph neural network and a second sub-graph neural network, and the first sub-graph neural network comprises the pre-trained prediction head, and further comprising:
extracting a second fingerprint generated by a second pre-trained prediction head of the second sub-graph neural network based on a second graph representation of the input compound; and
generating, utilizing a second neural network, the second fingerprint feature representation from the second fingerprint.

6. The computer-implemented method of claim 5, further comprising:
combining, utilizing a third neural network, the first fingerprint feature representation from the first sub-graph neural network and the second fingerprint feature representation from the second sub-graph neural network to generate the prediction for the second task corresponding to the input compound.

7. The computer-implemented method of claim 5, further comprising modifying parameters of the second neural network and a third neural network by comparing the prediction for the input compound with regard to the second task with a ground truth for the input compound with regard to the second task.

8. The computer-implemented method of claim 7, further comprising modifying the parameters of the second neural network and the third neural network while freezing parameters the pre-trained prediction head and the compound graph neural network.

9. The computer-implemented method of claim 1, wherein the pre-trained prediction head of the compound graph neural network is trained by:
generating, utilizing the pre-trained prediction head, a prediction for the first task from an additional graph representation of an additional input compound; and
modifying parameters of a prediction head by comparing the prediction for the first task with a ground truth for the first task.

10. A system comprising:
at least one processor; and at least one non-transitory computer-readable storage medium storing instructions that, when executed by the at least one processor to:
generate a graph representation reflecting node features and edge features from an input compound;
extract a fingerprint of the input compound generated from internal layers of a pre-trained prediction head of a compound graph neural network based on the graph representation of the input compound, wherein the pre-trained prediction head is trained to generate predictions for a first task;
generate, utilizing a neural network, a first fingerprint feature representation from the fingerprint; and
combine the first fingerprint feature representation and a second fingerprint feature representation to generate a prediction for the input compound with regard to a second task.

11. The system of claim 10, further comprising instructions that, when executed by the at least one processor, cause the system to:
extract a second fingerprint generated from internal layers of a second pre-trained prediction head of the compound graph neural network based on the graph representation of the input compound, wherein the second pre-trained prediction head is trained to generate predictions for a second task; and
generate, by a second neural network, the second fingerprint feature representation from the second fingerprint.

12. The system of claim 11, further comprising instructions that, when executed by the at least one processor, cause the system to:
combine, utilizing a third neural network, the first fingerprint feature representation and the second fingerprint feature representation to generate the prediction for the input compound with regard to the second task; and
modify parameters of the second neural network and the third neural network by comparing the prediction for the input compound with regard to the second task to a ground truth for the input compound with regard to the second task.

13. The system of claim 11, wherein the compound graph neural network comprises a graph-level pre-trained prediction head and a node-level pre-trained prediction head, wherein the second fingerprint extracted is a graph-level fingerprint from the graph-level pre-trained prediction head of the compound graph neural network.

14. The system of claim 10, wherein the compound graph neural network further comprises a first sub-graph neural network and a second sub-graph neural network, and the first sub-graph neural network further comprises the pre-trained prediction head, further comprising instructions that, when executed by the at least one processor, cause the system to:
extract a second fingerprint generated by a second pre-trained prediction head of the second sub-graph neural network based on a second graph representation of the input compound; and
generate, utilizing a second neural network, the second fingerprint feature representation from the second fingerprint.

15. The system of claim 10 further comprising instructions that, when executed by the at least one processor, cause the system to:
generate, utilizing the pre-trained prediction head, a prediction for the first task from an additional graph representation of an additional input compound; and
modify parameters of a prediction head by comparing the prediction for the first task with a ground truth for the first task.

16. A non-transitory computer-readable medium storing instructions that, when executed by at least one processor, cause a computing device to:
generate a graph representation reflecting node features and edge features from an input compound;
extract a fingerprint of the input compound generated from internal layers of a pre-trained prediction head of a compound graph neural network based on the graph representation of the input compound, wherein the pre-trained prediction head is trained to generate predictions for a first task;
generate, utilizing a neural network, a first fingerprint feature representation from the fingerprint; and
combine the first fingerprint feature representation and a second fingerprint feature representation to generate a prediction for the input compound with regard to a second task.

17. The non-transitory computer-readable medium of claim 16, further comprising instructions that, when executed by at least one processor, cause a computing device to:
extract a second fingerprint generated from internal layers of a second pre-trained prediction head of the compound graph neural network based on the graph representation of the input compound, wherein the second pre-trained prediction head is trained to generate predictions for a second task; and
generate, by a second neural network, the second fingerprint feature representation from the second fingerprint.

18. The non-transitory computer-readable medium of claim 17, further comprising instructions that, when executed by at least one processor, cause a computing device to:
combine, utilizing a third neural network, the first fingerprint feature representation and the second fingerprint feature representation to generate the prediction for the input compound with regard to the second task; and
modify parameters of the second neural network and the third neural network by comparing the prediction for the input compound with regard to the second task to a ground truth for the input compound with regard to the second task.

19. The non-transitory computer-readable medium of claim 17, wherein the compound graph neural network comprises a graph-level pre-trained prediction head and a node-level pre-trained prediction head, wherein the second fingerprint extracted is a graph-level fingerprint from the graph-level pre-trained prediction head of the compound graph neural network.

20. The non-transitory computer-readable medium of claim 16, wherein the compound graph neural network further comprises a first sub-graph neural network and a second sub-graph neural network, and the first sub-graph neural network further comprises the pre-trained prediction head, further comprising instructions that, when executed by at least one processor, cause a computing device to:
extract a second fingerprint generated by a second pre-trained prediction head of the second sub-graph neural network based on a second graph representation of the input compound; and
generate, utilizing a second neural network, the second fingerprint feature representation from the second fingerprint.
